(19) United States
(12) Patent Application Publication
(10) Pub. No.: US 2023/0310568 A1
(43) Pub. Date: Oct. 5, 2023

(54) ENGINEERED ARENAVIRUS GLYCOPROTEIN COMPOSITIONS AND METHODS OF USE THEREOF

(71) Applicants: La Jolla Institute for Immunology, La Jolla, CA (US); The Scripps Research Institute, La Jolla, CA (US)

(72) Inventors: Erica Ollmann Saphire, La Jolla, CA (US); Kathryn Hastie, La Jolla, CA (US); Haoyang Li, La Jolla, CA (US)

(21) Appl. No.: 18/021,792

(22) PCT Filed: Aug. 17, 2021
(86) PCT No.: PCT/US2021/046380

§ 371 Date: Feb. 16, 2023

Related U.S. Application Data
(60) Provisional application No. 63/066,644, filed on Aug. 17, 2020.

(51) Int. Cl. A61K 39/12; C07K 14/08; C12N 15/63; G01N 33/569

(52) U.S. Cl. CPC A61K 39/12; C07K 14/08; C12N 15/63; G01N 33/56983; C07K 2319/02; C12N 2760/10034; G01N 2469/20

(57) ABSTRACT

Provided herein are, inter alia, methods and compositions for treating and preventing arenavirus infection. Compositions include recombinant arenavirus glycoproteins that are able to form glycoprotein timers. The glycoprotein timers are contemplated to be effective for preventing and/or treating arenavirus infections.

### CROSS-REFERENCE TO RELATED APPLICATIONS

This application claims priority to U.S. Provisional Application No. 63/066,644, filed Aug. 17, 2020, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under R01AI13244 awarded by the National Institutes of Health. The government has certain rights in the invention.

### BACKGROUND

There is no available vaccine that protects people from infection with Lassa virus or most other pathogens in the Arenavirus family. The glycoprotein (GP) is the only antigen on the viral surface, and thus is a focus for vaccine design. The current reported vaccine candidates are unable to elicit neutralizing antibodies, likely because they failed to mimic the processed, pre-fusion state of the GP on the viral surface. Processed pre-fusion GP includes a trimer of dimers, each dimer including a non-covalently associated GP1 subunit and a GP2 subunit.

Furthermore, the largest and most potent group of neutralizing antibodies against LASV bind to quaternary epitopes involving adjacent GP monomers in the trimer, each in their prefusion conformation (1). A requisite for trimerization is proper processing of the GP. Large-scale production of the protein for commercialization necessitates the removal of stabilizing domains such as the stable signal peptide (SSP) and the GP transmembrane domain. The resulting ectodomain portions of GP do not remain associated with each another and the GP components separate and spring into the post-fusion form.

Therefore, generation of GP that mimic the native pre-fusion GP trimer of dimers is an essential point for vaccine design. Disclosed herein, inter alia, are solutions to these and other problems in the art.

### BRIEF SUMMARY OF THE INVENTION

In an aspect is provided a recombinant arenavirus glycoprotein including an arenavirus glycoprotein ectodomain and a trimerization domain.

In another aspect is provided a glycoprotein trimer including three of the recombinant arenavirus glycoproteins provided herein including embodiments thereof, wherein the three recombinant arenavirus glycoproteins are bound by non-covalent attachment of the trimerization domains.

In an aspect is provided a nucleic acid encoding a recombinant arenavirus glycoprotein provided herein including embodiments thereof.

In another aspect a cell is provided, the cell including a recombinant arenavirus glycoprotein provided herein including embodiments thereof or the glycoprotein trimer provided herein including embodiments thereof.

In an aspect is provided a cell including a nucleic acid provided herein including embodiments thereof.

In an aspect is provided a vaccine composition including the recombinant arenavirus glycoprotein provided herein including embodiments thereof and a pharmaceutically acceptable excipient.

In another aspect is provided a vaccine composition including the glycoprotein trimer provided herein including embodiments thereof and a pharmaceutically acceptable excipient.

In an aspect a method of treating or preventing a viral disease in a subject in need of such treatment or prevention is provided, the method including administering a therapeutically or prophylactically effective amount of a recombinant arenavirus glycoprotein provided herein including embodiments thereof to the subject.

In an aspect a method of treating or preventing a viral disease in a subject in need of such treatment or prevention is provided, the method including administering a therapeutically or prophylactically effective amount of a glycoprotein trimer provided herein including embodiments thereof to the subject.

In an aspect a method for immunizing a subject susceptible to a viral disease is provided, the method including administering a recombinant arenavirus glycoprotein provided herein including embodiments thereof to the subject under conditions such that antibodies directed to the arenavirus glycoprotein or a fragment thereof are produced.

In an aspect a method for immunizing a subject susceptible to a viral disease is provided, the method including administering a glycoprotein trimer provided herein including embodiments thereof to the subject under conditions such that antibodies directed to the glycoprotein trimer or a fragment thereof are produced.

In an aspect is provided a method of diagnosing arenavirus infection in a subject, the method including: (a) contacting a biological sample obtained from the subject with the recombinant arenavirus glycoprotein provided herein including embodiments thereof, and (b) detecting binding of one or more antibodies to said recombinant arenavirus glycoprotein, thereby diagnosing arenavirus infection in said subject.

In an aspect is provided a method of diagnosing arenavirus infection in a subject, the method including: (a) contacting a biological sample obtained from the subject with the glycoprotein trimer provided herein including embodiments thereof, and (b) detecting binding of one or more antibodies to said glycoprotein trimer, thereby diagnosing arenavirus infection in said subject.

In an aspect is provided a method for evaluating effectiveness of an arenavirus vaccine in a subject, the method including (a) contacting a biological sample from a subject who has been administered with the vaccine composition provided herein including embodiments thereof, (b) detecting antibodies in the biological sample that bind to the recombinant arenavirus glycoprotein or glycoprotein trimer provided herein including embodiments thereof, and (c) performing quantitative and qualitative analysis of the antibodies detected in the biological sample, thereby evaluating effectiveness of the arenavirus vaccine in the subject.

In an aspect is provided an antibody directed to a recombinant arenavirus glycoprotein provided herein including embodiments thereof.

In an aspect is provided an antibody directed to a glycoprotein trimer provided herein including embodiments thereof.

In an aspect a method of generating arenavirus-specific antibodies is provided, the method including administering any of the compositions provided herein including embodiments thereof to a subject, obtaining biological material from the subject, and purifying antibodies from the biological material.

In an aspect a method for detecting arenavirus infection is provided, the method including contacting a biological sample with an antibody provided herein, and detecting the presence or absence of arenavirus.

A method of determining the presence of antibodies specific for an arenavirus in a biological sample including contacting the biological sample with a composition including a recombinant arenavirus glycoprotein provided herein, and detecting the presence or absence of arenavirus-specific antibody.

### BRIEF DESCRIPTION OF THE DRAWINGS

FIG. 1A is a schematic showing generation of pre-fusion GP trimer of arenaviruses by fused expression of the ectodomain of GPCysR4 with a heterologous C-terminal trimerization domain.

FIG. 1B is a schematic showing generation of pre-fusion GP trimer of arenaviruses by expressing the GPCysR4-LPXTG monomer and the heterologous trimer separately, and covalently joined by the sortase-mediated transpeptidation to form GPCysR4-LPXTG-TD trimers.

FIG. 2 is a graph showing antibody response to unmodified LASV GP (called GPmper) and the prefusion-trimeric GP (GPTD).

FIGS. 3A and 3B are images of a non-reducing SDS-PAGE of purified LASV GPcysR4-INOG (FIG. 3A) and an electron micrograph of the LASV GPcysR4-1NOG/37.2D Fab trimers by negative stain (FIG. 3B).

FIGS. 4A and 4B illustrate results of the purification of GPCysR4-LPETG-1NOG. A chromatogram shows the product of sortase A-mediated LASV GPCysR4-LPETG-1NOG formation purified by size exclusion chromatography using a S200Inc size exclusion column (FIG. 4A). An image of a non-reducing SDS-PAGE shows molecular weight analysis of different peaks collected from the size exclusion column (FIG. 4B).

FIGS. 5A and 5B are images of the negative stain micrographs of sortase-derived LASV GPCysR4-LPETG-1NOG trimers (FIG. 5A) and sortase derived LASV GPCysR4-LPETG-1NOG /37.2D Fab trimers (FIG. 5B).

FIG. 6 is a schematic showing the native pre-fusion Lassa GP trimer, which is able to elicit neutralizing antibodies.

FIG. 7 are schematics showing embodiments of the trimerized recombinant arenavirus glycoproteins described herein. The trimerized Lassa GP immunogens mimic native pre-fusion Lassa GP.

FIGS. 8A-8C show optimization of cleavage sites for various expression systems. Schematics illustrate the GP1/GP2 protein with different cleavage sites designed for insect cell (top panel) or mammalian cell (bottom panel) expression systems (FIG. 8A). Representative images of SDS PAGE gels show that GPCysRRLL can be cleaved in 293F cells and GPCysR4 can be cleaved in S2 cells (FIG. 8B), and GPCysR4 can't be cleaved in 293F cells (FIG. 8C).

FIG. 9 shows 3D maps of negative stain electron microscopy (NS-EM) of Lassa GPCysR4-INOG trimer and Lujo GPCysR4-INOG trimer. The 3D maps show that both Lassa and Lujo GPCysR4-INOGs are able to form pre-fusion GP trimers.

FIGS. 10A-10D show that Lassa GPCysR4-INOG trimer is recognized by anti-Lassa neutralizing antibodies. A NS-EM 3D-map of Lassa GPCysR4-INOG timer shows that the 37.2D Fab complexes with the timer (FIG. 10A). NS-EM micrographs of Lassa GPCysR4-INOG trimer shows that the trimer complexes with 25.10C Fabs (FIG. 10B), 12.1F Fabs (FIG. 10C), and 37.7H Fabs (FIG. 10D).

FIGS. 11A-11C illustrate expression and purification of a GPCysR4 monomer and INOG trimer separately for generating a fully processed Lassa GP trimer. A representative image of an SDS PAGE gel shows incomplete cleavage of the Lassa GPCysR4-INOG protein into GP1 and GP2-INOG. Results show that >90% Lassa GPCysR4-INOG proteins are cleaved, leaving about 10% Lassa GPCysR4-INOG fusion proteins unprocessed (uncleaved) (FIG. 11A). A peptide linker with a sortase recognition site is used for fusing the GPCysR4 and INOG into a pre-fusion timer (FIG. 11B), thereby generating a 100% processed GP trimer (FIG. 11C).

FIGS. 12A and 12B are ribbon diagrams showing the structural configuration of the INOG trimerization domain. The top view of the trimerization domain is shown (FIG. 12A, top panel) in the glycoprotein trimer, illustrating the triangular configuration of the N-terminus (FIG. 12A, bottom panel), which is attached to the C-terminus of the GP2 domain. The C-terminus view timer is shown (FIG. 12B), illustrating the structural configuration of trimerization domain from the bottom view of the glycoprotein timer.

### DETAILED DESCRIPTION

The recombinant arenavirus glycoprotein provided herein, including embodiments thereof, include an arenavirus glycoprotein ectodomain and a trimerization domain. The arenavirus glycoproteins form glycoprotein trimers by non-covalently binding each other through trimerization domains. The glycoprotein trimers are recognized by neutralizing antibodies. Thus, compositions including the recombinant arenavirus glycoprotein and or glycoprotein trimers are contemplated to be effective for treating and/or preventing arenavirus infection and associated diseases.

Compositions including the recombinant arenavirus glycoprotein and or glycoprotein trimers may further be used in antibody discovery. The compositions may be used as in diagnostic methods to characterize antibody response upon natural infection or vaccination. The compositions are further contemplated to be useful for characterizing the structure of arenavirus proteins and are useful for small molecule drug design targeting exogenous glycoprotein domains.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

A “chemical linker,” as provided herein, is a covalent linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered,) heteroarylene.

In embodiments, the chemical linker is a covalent linker. In embodiments, the chemical linker is a hydrocarbon linker.

Thus, a chemical linker as provided herein may include a plurality of chemical moieties, wherein each of the plurality of chemical moieties is chemically different. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one defmition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “lassavirus mammarenavirus glycoprotein” or “LASV GP” as provided herein includes any of the recombinant or naturally-occurring forms of lassavirus mammarenavirus glycoprotein (LASV GP), also known as Pre-glycoprotein polyprotein GP complex, Pre-GP-C, or variants or homologs thereof that maintain LASV GP activity (e.g. within at least 50%, 80%, 90%, 95%, 96%, 97%, 98%, 99% or 100% activity compared to LASV GP). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LASV GP protein polypeptide. In embodiments, LASV GP protein is the protein as identified by the UniProt reference number P08669, or a variant, homolog or functional fragment thereof. In aspects, LASV GP includes the amino acid sequence of SEQ ID NO:13. In aspects, LASV GP has the amino acid sequence of SEQ ID NO:13. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:13. In aspects, LASV GP includes the amino acid sequence of SEQ ID NO:35. In aspects, LASV GP has the amino acid sequence of SEQ ID NO:35. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:35. In aspects, LASV GP includes the amino acid sequence of SEQ ID NO:36. In aspects, LASV GP has the amino acid sequence of SEQ ID NO:36. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:36. In aspects, LASV GP includes the amino acid sequence of SEQ ID NO:37. In aspects, LASV GP has the amino acid sequence of SEQ ID NO:37. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:37. In aspects, LASV GP includes the amino acid sequence of SEQ ID NO:38. In aspects, LASV GP has the amino acid sequence of SEQ ID NO:38. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:38. In aspects, LASV GP includes the amino acid sequence of SEQ ID NO:39. In aspects, LASV GP has the amino acid sequence of SEQ ID NO:39. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:39. In aspects, LASV GP includes the amino acid sequence of SEQ ID NO:40. In aspects, LASV GP has the amino acid sequence of SEQ ID NO:40. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:40. In aspects, LASV GP includes the amino acid sequence of SEQ ID NO:41. In aspects, LASV GP has the amino acid sequence of SEQ ID NO:41. In aspects, LASV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:41.

The term “Lymphocytic choriomeningitis virus glycoprotein” or “LCMV GP” as provided herein includes any of the recombinant or naturally-occurring forms of Lymphocytic choriomeningitis virus glycoprotein (LCMV GP), also known as Pre-glycoprotein polyprotein GP complex, Pre-GP-C, or variants or homologs thereof that maintain LCMV GP activity (e.g. within at least 50%, 80%, 90%, 95%, 96%, 97%, 98%, 99% or 100% activity compared to LCMV GP). In aspects, the variants or homologs have at least 90%, 95%, 96%, 97%, 98%, 99% or 100% amino acid sequence identity across the whole sequence or a portion of the sequence e.g. a 50, 100, 150 or 200 continuous amino acid portion) compared to a naturally occurring LCMV GP polypeptide. In embodiments, LCMV GP is the protein as identified by the UniProt reference number P09991, or a variant, homolog or functional fragment thereof. In aspects, LCMV GP includes the amino acid sequence of SEQ ID NO:42. In aspects, LCMV GP has the amino acid sequence of SEQ ID NO:42. In aspects, LCMV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:42.

The term “Lujo virus glycoprotein” or “Lujo virus GP” as provided herein includes any of the recombinant or naturally-occurring forms of Lujo virus glycoprotein (Lujo virus GP), also known as Pre-glycoprotein polyprotein GP complex, Pre-GP-C, or variants or homologs thereof that maintain Lujo virus GP activity (e.g. within at least 50%, 80%, 90%, 95%, 96%, 97%, 98%, 99% or 100% activity compared to Lujo virus GP). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Lujo virus GP polypeptide. In embodiments, Lujo virus GP is the protein as identified by the UniProt reference number C5ILC1, or a variant, homolog or functional fragment thereof. In aspects, Lujo virus GP includes the amino acid sequence of SEQ ID NO:43. In aspects, Lujo virus GP has the amino acid sequence of SEQ ID NO:43. In aspects, Lujo virus GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:43.

The term “Machupo virus glycoprotein” or “MACV GP” as provided herein includes any of the recombinant or naturally-occurring forms of Machupo virus glycoprotein (MACV GP), also known as Pre-glycoprotein polyprotein GP complex, Pre-GP-C, or variants or homologs thereof that maintain MACV GP activity (e.g. within at least 50%, 80%, 90%, 95%, 96%, 97%, 98%, 99% or 100% activity compared to MACV GP). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MACV GP polypeptide. In embodiments, MACV GP is the protein as identified by the UniProt reference number Q6IUF7, or a variant, homolog or functional fragment thereof. In aspects, MACV GP includes the amino acid sequence of SEQ ID NO:44. In aspects, MACV GP has the amino acid sequence of SEQ ID NO:44. In aspects, MACV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:44.

The term “Junin mammarenavirus glycoprotein” or “JUNV GP” as provided herein includes any of the recombinant or naturally-occurring forms of Junin mammarenavirus glycoprotein (JUNV GP), also known as Pre-glycoprotein polyprotein GP complex, Pre-GP-C, or variants or homologs thereof that maintain JUNV GP activity (e.g. within at least 50%, 80%, 90%, 95%, 96%, 97%, 98%, 99% or 100% activity compared to JUNV GP). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring JUNV GP polypeptide. In embodiments, JUNV GP is the protein as identified by the UniProt reference number P26313, or a variant, homolog or functional fragment thereof. In aspects, JUNV GP includes the amino acid sequence of SEQ ID NO:45. In aspects, JUNV GP has the amino acid sequence of SEQ ID NO:45. In aspects, JUNV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:45.

The term “fibritin” or “fibritin protein” as provided herein includes any of the recombinant or naturally-occurring forms of fibritin, also known as Collar protein, Whisker antigen control protein, or variants or homologs thereof that maintain fibritin activity (e.g. within at least 50%, 80%, 90%, 95%, 96%, 97%, 98%, 99% or 100% activity compared to fibritin). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring fibritin protein polypeptide. In embodiments, fibritin is the protein as identified by the UniProt reference number P10104, or a variant, homolog or functional fragment thereof.

The term “General control transcription factor GCN4” or “General control transcription factor GCN4 protein” as provided herein includes any of the recombinant or naturally-occurring forms of General control transcription factor GCN4 (GCN4), also known as Amino acid biosynthesis regulatory protein, General control protein GCN4, or variants or homologs thereof that maintain GCN4 protein activity (e.g. within at least 50%, 80%, 90%, 95%, 96%, 97%, 98%, 99% or 100% activity compared to GCN4 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GCN4 protein polypeptide. In embodiments, GNC4 is the protein as identified by the UniProt reference number P03069, or a variant, homolog or functional fragment thereof.

As used interchangeably herein, the terms “fusion protein” and “fusion polypeptide” refer to a polypeptide or an amino acid sequence linked to at least a second polypeptide or an amino acid sequence derived from a second polypeptide. The individualized elements of the fusion protein can be linked in any of a variety of ways, including for example, direct attachment, the use of an intermediate or spacer peptide, or the use of a linker region. In embodiments, the linker region is a covalent bond or a peptide linker. For example, the linker peptide includes anywhere from 0 to 100 amino acids, from 0 to 90 amino acids, from 0 to 80 amino acids, from 0 to 70 amino acids, from 0 to 60 amino acids, from 0 to 50 amino acids, from 0 to 55 amino acids, from 0 to 40 amino acids, from 0 to 35 amino acids, from 0 to 30 amino acids, from 0 to 25 amino acids, from 0 to 20 amino acids, from 0 to 15 amino acids, from 0 to 10 amino acids, 0 zero to 5 amino acids, or 0 zero to 3 amino acids. In embodiments, the polypeptide (e.g. GP1) is linked to a second polypeptide (e.g. GP2) by way of a covalent bond (e.g. a peptide bond). In embodiments, the polypeptide is linked to a second polypeptide by one or more disulfide linkages. For example, a polypeptide (e.g. GP1) may include a first cysteine amino acid side chain which may form a disulfide bond with a second cysteine amino acid side chain in a second polypeptide (e.g. GP2), thereby forming a fusion protein. Thus, in embodiments, the fusion protein includes two polypeptides (e.g. GP1 and GP2) covalently attached by way of one or more disulfide bonds. Thus, in embodiments, the fusion protein is a non-linear polypeptide.

The term “glycoprotein” or “GP” refers to proteins that include oligosaccharides covalently attached to amino acid side-chains. In embodiments, the GP is a Lassa mammarenavirus (LASV) GP. In embodiments, the GP is a Lymphocytic Choriomeningitis (LCM) GP. In embodiments, the GP is a Lujo virus GP. In embodiments, the GP is a Machupo virus GP. In embodiments, the GP is a Junin virus GP. The glycoprotein of Arenaviridae, is synthesized as precursor protein pre-GP-C and is typically cotranslationally cleaved by signal peptidase into GP-C and the signal peptide, which in instances exhibits unusual length, stability, and topology. In embodiments, the mature GPC is a trimer of heterodimers composed of the non-covalently associated subunits GP1 and GP2. In embodiments, the mature GPC is a trimer of heterotrimers composed of the non-covalently associated subunits GP1 and GP2, and SSP. SSP is the transmembrane stable signal peptide and is thought to be involved in viral infectivity. In instances, GP1 binds receptor and determines tropism. In instances, GP2 may drive fusion of virus and host membranes, wherein GP2 undergoes an acid pH-driven, conformational change from a metastable, prefusion structure to a more stable, postfusion, six-helix bundle. Typically, after processing by signal peptidase, GP-C of both New World and Old World arenaviruses are cleaved by the cellular subtilase subtilisin kexin isozyme-1/site-1 protease (SKI-1/S1P) into the distal subunit GP-1 and the membrane-anchored subunit GP-2 within the secretory pathway.

TABLE 1
Sequences and characteristics of Lassa virus glycoproteins

| Lassa virus GP | Full length | Ectodomain | Genebank NO. |
|---|---|---|---|
| Lineage 1 | 490aa | 59-431aa | AAF86701.1 |
| Lineage 2 | 490aa | 59-431aa | AVO03605.1 |
| Lineage 3 | 490aa | 59-431aa | AVO03595.1 |
| Lineage 4 | 491aa | 59-432aa | NP_694870.1 |
| Lineage 5 | 491aa | 59-432aa | AHC95557.1 |
| Lineage 6 | 490aa | 59-431aa | AMR44577.1 |
| Lineage 7 | 490aa | 59-431aa | ANH09740.1 |

TABLE 2
Sequences and characteristics of Arenavirus glycoproteins

| Arenavirus GP | Full length | Ectodomain | Genebank NO. |
|---|---|---|---|
| Lassa virus (Josiah strain) | 491aa | 59-432aa | NP_694870.1 |
| Lymphocytic choriomeningitis | 498aa | 59-438aa | ABC96001.2 |
| virus (Clone 13 strain) | | | |
| Lujo virus | 454aa | 59-394aa | AFP21514.1 |
| Machupo virus | 496aa | 59-435aa | AMZ00396.1 |
| Junin virus | 485aa | 59-424aa | AAB65463.1 |

“GPCysR4” refers to a genetically modified the LASV glycoprotein ectodomain. GPCysR4 includes R207C and G360C point mutations, which allow disulfide bond formation between GP1 and GP2 together. GPCysR4 includes a E329P mutation in the metastable region of HR1 of GP2. GPCysR4 further includes a modification that includes replacment of the native S113 GP1-GP2 cleavage site with a furin site to enable efficient processing of the GP in Drosophila S2 cells.

As used interchangeably herein, the terms “signal peptide” and “signal sequence” refer to a protein or peptide sequence that enables a cell to translocate a protein. In embodiments, the protein is translocated to the cell membrane.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab2, bispecific Fab2, trispecific Fab3, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibodyvariants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VII with the C-terminus of the VL, or vice versa.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “multimer” refers to a complex comprising multiple monomers (e.g. a protein complex) associated by noncovalent bonds. The monomers be substantially identical monomers, or the monomers may be different. In embodiments, the multimer is a dimer, a trimer, a tetramer, or a pentamer. Thus, a trimer comprises three monomers associated by noncovalent bonds.

A “ligand” refers to an agent, e.g., a polypeptide or other molecule, capable of binding to a receptor or antibody, antibody variant, antibody region or fragment thereof.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The terms “virus” or “virus particle” are used according to their plain ordinary meaning in the biological arts and refer to a particle including a viral genome (e.g. DNA, RNA, single strand, double strand), a protective coat of proteins (e.g. capsid) and associated proteins, and in the case of enveloped viruses (e.g. herpesvirus), an envelope including lipids and optionally components of host cell membranes, and/or viral proteins. In embodiments, the virus is an Arenavirus.

The terms “multiplicity of infection” or “MOI” are used according to its plain ordinary meaning in Virology and refers to the ratio of components to the target (e.g., cell) in a given area. In embodiments, the area is assumed to be homogenous.

“Arenavirus” refers to a member of the group of single stranded, negative sense RNA viruses with two nucleic acid segments that are members of the family Arenaviridae. The bisegmented single-stranded RNA genome of Arenaviruses encode the polymerase L, matrix protein Z, nucleoprotein NP, and glycoprotein GP. The bipartite ribonucleoprotein of LASV is typically surrounded by a lipid envelope derived from the plasma membrane of the host cell. The matrix protein Z has been identified as a major budding factor, which lines the interior of the viral lipid membrane, in which GP spikes are inserted. Arenaviruses may infect animals, for example rodents and snakes, and some infect humans to cause disease. Arenaviruses may acquire ribosomes from their host cells. Lassa virus (LASV) is a member of the family Arenaviridae, of which Lymphocytic choriomeningitis virus (LCMV) is the prototype. Arenaviruses comprise more than 20 species, divided into the Old World and New World virus complexes. The Old World arenaviruses include the human pathogenic LASV strains, Lujo virus, which was first identified in late 2008 and is associated with an unprecedented high case fatality rate in humans, the nonhuman pathogenic Ippy, Mobala, and Mopeia viruses, and the recently described Kodoko virus. The New World virus complex contains, among others, the South American hemorrhagic fever-causing viruses Junin virus, Machupo virus, Guanarito virus, Sabia virus, and the recently discovered Chapare virus. Thus, in embodiments, the arenavirus is LASV, LCMV, Junin virus, Machupo virus, Guanarito virus, Sabia virus, or Chapare virus.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods. Thus, a recombinant protein refers to a protein made by introducing a cell with a nucleic acid that is not typically found in the cell (e.g. non-native DNA). The cells containing the non-native nucleic acid may then transcribe and translate the protein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES,

Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

As defined herein, the term “inhibition”, “inhibit”, “inhibiting” and the like in reference to cell proliferation (e.g., cancer cell proliferation) means negatively affecting (e.g., decreasing proliferation) or killing the cell. In embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g.,, by binding, partially or totally blocking, decreasing, preventing, delaying, inactivating, desensitizing, or down-regulating activity (e.g., a receptor activity or a protein activity).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein.

The term “associated” or “associated with” in the context of a substance or substance activity or function associated with a disease (e.g., arenavirus infection) means that the disease is caused by (in whole or in part), or a symptom of the disease is caused by (in whole or in part) the substance or substance activity or function. Alternatively, the substance may be an indicator of the disease. Thus, an associated substance may serve as a means of targeting disease tissue.

A “therapeutic agent” as referred to herein, is a composition useful in treating or preventing a disease such as a viral infection (e.g. Lassa fever). In embodiments, the therpaeutic agent is an anti-viral agent. “Anti-viral agent” is used in accordance with its plain ordinary meaning and refers to a composition (e.g. compound, drug, antagonist, inhibitor, modulator) having anti-viral properties or the ability to inhibit viral infection. In embodiments, an anti-viral agent targets a viral protein. In embodiments, an anti-viral agent inhibits viral entry into a host cell. In embodiments, an anti-viral agent inhibits replication of viral components. In embodiments, an anti-viral inhibits release of viral particles. In embodiments, an anti-viral inhibits assembly of viral particles.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

As used herein, a “symptom” of a disease includes any clinical or laboratory manifestation associated with the disease, and is not limited to what a subject can feel or observe.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

Pharmaceutical compositions can also include large, slowly metabolized macromolecules such as proteins, polysaccharides such as chitosan, polylactic acids, polyglycolic acids and copolymers (such as latex functionalized sepharose™, agarose, cellulose, and the like), polymeric amino acids, amino acid copolymers, and lipid aggregates such as oil droplets or liposomes). Additionally, these carriers can function as immunostimulating agents (i.e., adjuvants).

The term “adjuvant” refers to a compound that when administered in conjunction with the agents provided herein including embodiments thereof, augments the agent's immune response. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

### Arenavirus Glycoprotein Compositions

The recombinant arenavirus glycoprotein provided herein including embodiments thereof include an arenavirus glycoprotein ectodomain and a trimerization domain. As used herein, “ectodomain” refers to the portion or fragment of a protein that is on the surface of a cell or virus particle. For example, the ectodomain of an arenavirus glycoprotein includes portions of Glycoprotein1 (GP1) and Glycoprotein2 (GP2) which remain on the surface of the viral particle. Thus, an arenavirus glycoprotein ectodomain lacks the portions of GP1 and GP2 which are in the viral lipid membrane (e.g. transmembrane domain). In instances, the ectodomain includes a membrane proximal region. For example, in instances, GP2 includes a membrane proximal region having the amino acid sequence of SEQ ID NO:4. The recombinant arenavirus glycoprotein provided herein including embodiments thereof includes a trimerization domain. As used herein, the term “trimerization domain” refers to a protein or peptide domain that is capable of non-covalently binding two other trimerization domains to form a protein trimer. As used herein, trimerization domain refers to a protein that is not naturally encoded by the arenavirus genome (e.g. an exogenous trimerization domain). Thus, in an aspect is provided a recombinant arenavirus glycoprotein including an arenavirus glycoprotein ectodomain and a trimerization domain.

In embodiments, the arenavirus glycoprotein ectodomain includes the amino acid sequence of SEQ ID NO:3. In embodiments, the arenavirus glycoprotein ectodomain is the amino acid sequence of SEQ ID NO:3. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:3. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:3.

In embodiments, the arenavirus glycoprotein ectodomain includes the amino acid sequence of SEQ ID NO:27. In embodiments, the arenavirus glycoprotein ectodomain is the amino acid sequence of SEQ ID NO:27. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:27. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:27.

In embodiments, the arenavirus glycoprotein ectodomain includes the amino acid sequence of SEQ ID NO:35. In embodiments, the arenavirus glycoprotein ectodomain is the amino acid sequence of SEQ ID NO:35. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:35. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:35.

In embodiments, the arenavirus glycoprotein ectodomain includes the amino acid sequence of SEQ ID NO:36. In embodiments, the arenavirus glycoprotein ectodomain is the amino acid sequence of SEQ ID NO:36. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:36. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:36.

In embodiments, the arenavirus glycoprotein ectodomain includes the amino acid sequence of SEQ ID NO:37. In embodiments, the arenavirus glycoprotein ectodomain is the amino acid sequence of SEQ ID NO:37. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:37. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:37.

In embodiments, the arenavirus glycoprotein ectodomain includes the amino acid sequence of SEQ ID NO:38. In embodiments, the arenavirus glycoprotein ectodomain is the amino acid sequence of SEQ ID NO:38. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:38. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:38.

In embodiments, the arenavirus glycoprotein ectodomain includes the amino acid sequence of SEQ ID NO:39. In embodiments, the arenavirus glycoprotein ectodomain is the amino acid sequence of SEQ ID NO:39. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:39. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:39.

In embodiments, the arenavirus glycoprotein ectodomain includes the amino acid sequence of SEQ ID NO:40. In embodiments, the arenavirus glycoprotein ectodomain is the amino acid sequence of SEQ ID NO:40. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:40. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:40.

In embodiments, the arenavirus glycoprotein ectodomain includes the amino acid sequence of SEQ ID NO:41. In embodiments, the arenavirus glycoprotein ectodomain is the amino acid sequence of SEQ ID NO:41. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:41. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:41.

In embodiments, the arenavirus glycoprotein ectodomain includes the amino acid sequence of SEQ ID NO:42. In embodiments, the arenavirus glycoprotein ectodomain is the amino acid sequence of SEQ ID NO:42. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:42. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:42.

In embodiments, the arenavirus glycoprotein ectodomain includes the amino acid sequence of SEQ ID NO:42. In embodiments, the arenavirus glycoprotein ectodomain is the amino acid sequence of SEQ ID NO:42. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:42. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:42.

In embodiments, the arenavirus glycoprotein ectodomain includes the amino acid sequence of SEQ ID NO:43. In embodiments, the arenavirus glycoprotein ectodomain is the amino acid sequence of SEQ ID NO:43. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:43. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:43.

In embodiments, the arenavirus glycoprotein ectodomain includes the amino acid sequence of SEQ ID NO:44. In embodiments, the arenavirus glycoprotein ectodomain is the amino acid sequence of SEQ ID NO:44. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:44. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:44.

In embodiments, the arenavirus glycoprotein ectodomain includes the amino acid sequence of SEQ ID NO:45. In embodiments, the arenavirus glycoprotein ectodomain is the amino acid sequence of SEQ ID NO:45. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:45. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:45.

In embodiments, the ectodomain includes an arenavirus GP1/GP2 protein, wherein the arenavirus GP1/GP2 protein includes a GP1 domain and a GP2 domain. As used herein, “GP1/GP2 protein” refers to ectodomain portions of the GP1 domain and the GP2 domain which are bound to each other by way of covalent bonds. For example, the N-terminus of GP2 may be attached to the C-terminus of GP1. Thus, in embodiments, the GP1/GP2 protein is a linear polypeptide. In embodiments, the GP1 and GP2 are bound to each other by way of one or more disulfide bonds formed between one or more cysteine amino acid side chains in the GP1 domain and one or more cysteine amino acid side chains in the GP2 domain.

In embodiments, the GP1 domain includes an amino acid sequence having at least 80%, 85%, 90%, 91%, 92%, 93%, 94%, 95%, 96%, 97%, 98%, or 99% sequence identify to SEQ ID NO:28. In embodiments, GP1 includes an amino acid sequence having at least 80% sequence identify to SEQ ID NO:28. In embodiments, GP1 includes an amino acid sequence having at least 85% sequence identify to SEQ ID NO:28. In embodiments, GP1 includes an amino acid sequence having at least 90% sequence identify to SEQ ID NO:28. In embodiments, GP1 includes an amino acid sequence having at least 95% sequence identify to SEQ ID NO:28. In embodiments, GP1 includes an amino acid sequence having at least 98% sequence identify to SEQ ID NO:28. In embodiments, GP1 includes the amino acid sequence of SEQ ID NO:28. In embodiments, GP1 is the amino acid sequence of SEQ ID NO:28.

In embodiments, GP2 includes an amino acid sequence having at least 80%, 85%, 90%, 91%, 92%, 93%, 94%, 95%, 96%, 97%, 98%, or 99% sequence identify to SEQ ID NO:29. In embodiments, GP2 includes an amino acid sequence having at least 80% sequence identify to SEQ ID NO:29. In embodiments, GP2 includes an amino acid sequence having at least 85% sequence identify to SEQ ID NO:29. In embodiments, GP2 includes an amino acid sequence having at least 90% sequence identify to SEQ ID NO:29. In embodiments, GP2 includes an amino acid sequence having at least 95% sequence identify to SEQ ID NO:29. In embodiments, GP2 includes an amino acid sequence having at least 98% sequence identify to SEQ ID NO:29. In embodiments, GP2 includes the amino acid sequence of SEQ ID NO:29. In embodiments, GP2 is the amino acid sequence of SEQ ID NO:29.

In embodiments, the C-terminus of the GP1 domain is bound to the N-terminus of the GP2 domain. In instances, the C-terminus of the GP1 domain is bound to the N-terminus of the GP2 domain by way of a covalent bond (e.g. a peptide bond). In instances, the C-terminus of the GP1 domain is bound to the N-terminus of the GP2 domain through a peptide linker. In 30 embodiments, the peptide linker includes C-terminus residues of the GP1 domain and N-terminus residues of the GP2 domain. For example, the peptide linker may include 1, 2, 3, 4, 5, 6, 7, 8, 9 or 10 residues from the C-terminus of the GP1 domain. For example, the peptide linker may include 1, 2, 3, 4, 5, 6, 7, 8, 9 or 10 residues from the N-terminus of the GP2 domain. In instances, the C-terminus of the GP1 domain is bound to the N-terminus of the GP2 domain through a chemical linker. In embodiments, the GP1/GP2 protein is recombinantly expressed as a single moiety. In embodiments, the GP1/GP2 protein is expressed as separate moieties which are then bound to each other by covalent methods. Methods for covalently linking polypeptides include those well known in the art and described herein. For example, chemical linkers, peptide linkers, and bioconjugate linkers as described herein may be used for covalently linking GP1 to GP2, thereby forming the the GP1/GP2 protein.

For the recombinant arenavirus glycoprotein provided herein, in embodiments, the ectodomain further includes a protease cleavage site, wherein the protease cleavage site is located between said GP1 domain and said GP2 domain. In embodiments, the protease cleavage site is within a peptide linker attaching the C-terminus of the GP1 domain to the N-terminus of the GP2 domain.

A “protease cleavage site” as used herein, refers to a recognizable site for cleavage of a portion of polypeptide provided herein including embodiments thereof. Thus, a cleavage site may be found in the sequence of a polypeptide as described herein, including embodiments thereof. Thus, in embodiments, the cleavage site is an amino acid sequence that is recognized and cleaved by a cleaving agent (e.g., a protease). In embodiments, the protease cleavage site includes the sequence of SEQ ID NO:6, 9, 19, 20, 21, 22, 23, 24, 25, 26, or 30. Any protease cleavage site known in the art for cleaving viral glycoproteins may be used. Protease cleavage sites suitable for cleaving viral glycoproteins are described in greater detail in Burri, D. J. et al. Differential Recognition of Old World and New World Arenavirus Envelope Glycoproteins by Subtilisin Kexin Isozyme 1 (SKI-1)/Site 1 Protease (S1P). J. Virol. Volume 87, Issue 11, 1 June 2013, Pages 6406-6414; https://doi.org/10.1128/JVI.00072-13.; which is incorporated by reference herein in its entirety and for all purposes.

In embodiments, the arenavirus GP1/GP2 protein is a stabilized arenavirus GP1/GP2 protein. “Stabilized arenavirus GP1/GP2 protein” refers to a GP1/GP2 protein, wherein GP1 and GP2 are more likely to form a complex as compared to a wild type GP1/GP2. For example, GP1 and GP2 in a stabilized GP1/GP2 protein are more likely to form a complex after cleavage of a covalent bond (e.g. peptide bond) attaching the C-terminus of GP1 to the N-terminus of GP2. For instance, the stabilized arenavirus GP1/GP2 protein may include amino acid substitutions which are contemplated to increase formation of a GP1/GP2 complex after cleavage of a covalent bond attaching GP1 to GP2. In instances, the stabilized arenavirus GP1/GP2 protein may include amino acid substitutions which allow non-covalent interactions between GP1 and GP2. For example, the GP1 domain and GP2 domain may include amino acid substitutions which allow electrostatic interactions (e.g. ionic bond, hydrogen bond, halogen bond) or hydrophobic interactions between GP1 and GP2, thereby increasing formation of the GP1/GP2 complex. In embodiments, GP1 and GP2 may include cysteine point mutations, thereby allowing disulfide bond formation between a first cysteine amino acid side chain in GP1 and a second cysteine amino acid side chain in GP2. Thus, in embodiments, the stabilized arenavirus GP1/GP2 protein includes a disulfide bond between a first cysteine amino acid side chain in the GP1 domain and a second cysteine amino acid side chain in the GP2 domain. In embodiments, the first cysteine amino acid side chain in the GP1 domain is at a position corresponding to position 207 of SEQ ID NO:18 and the second cysteine amino acid side chain in the GP2 domain is at a position corresponding to position 360 of SEQ ID NO:18. In embodiments, the first cysteine amino acid side chain in the GP1 domain is at a position corresponding to position 243 of SEQ ID NO:18 and the second cysteine amino acid side chain in the GP2 domain is at a position corresponding to position 350 of SEQ ID NO:18.

For the recombinant arenavirus glycoprotein provided herein including embodiments thereof, the trimerization domain is bound to the C-terminus of said GP2 domain.

In embodiments, the trimerization domain is covalently bound to the C-terminus of the GP2 domain though a chemical linker. In embodiments, the trimerization domain is covalently bound to the C-terminus of the GP2 domain though a peptide linker. In embodiments, the peptide linker includes 1, 2, 3, 4, 5, 6, 7, 8, 9 or 10 C-terminal amino acid residues of the GP2 domain. In embodiments, the peptide linker includes 1, 2, 3, 4, 5, 6, 7, 8, 9 or 10 N-terminal amino acid residues of the trimerization domain. In embodiments, the peptide linker includes the amino acid sequence of SEQ ID NO:32. In embodiments, the peptide linker includes the amino acid sequence of SEQ ID NO:33. In embodiments, the peptide linker includes the amino acid sequence of SEQ ID NO:34.

In embodiments, the peptide linker is from about 5 to about 80 amino acid residues in length. In embodiments, the peptide linker is from about 10 to about 80 amino acid residues in length. In embodiments, the peptide linker is from about 15 to about 80 amino acid residues in length. In embodiments, the peptide linker is from about 20 to about 80 amino acid residues in length. In embodiments, the peptide linker is from about 25 to about 80 amino acid residues in length. In embodiments, the peptide linker is from about 30 to about 80 amino acid residues in length. In embodiments, the peptide linker is from about 35 to about 80 amino acid residues in length. In embodiments, the peptide linker is from about 40 to about 80 amino acid residues in length. In embodiments, the peptide linker is from about 45 to about 80 amino acid residues in length. In embodiments, the peptide linker is from about 50 to about 80 amino acid residues in length. In embodiments, the peptide linker is from about 55 to about 80 amino acid residues in length. In embodiments, the peptide linker is from about 60 to about 80 amino acid residues in length. In embodiments, the peptide linker is from about 65 to about 80 amino acid residues in length. In embodiments, the peptide linker is from about 70 to about 80 amino acid residues in length. In embodiments, the peptide linker is from about 75 to about 80 amino acid residues in length.

In embodiments, the peptide linker is from about 5 to about 75 amino acid residues in length. In embodiments, the peptide linker is from about 5 to about 70 amino acid residues in length. In embodiments, the peptide linker is from about 5 to about 65 amino acid residues in length. In embodiments, the peptide linker is from about 5 to about 60 amino acid residues in length. In embodiments, the peptide linker is from about 5 to about 55 amino acid residues in length. In embodiments, the peptide linker is from about 5 to about 50 amino acid residues in length. In embodiments, the peptide linker is from about 5 to about 45 amino acid residues in length. In embodiments, the peptide linker is from about 5 to about 40 amino acid residues in length. In embodiments, the peptide linker is from about 5 to about 35 amino acid residues in length. In embodiments, the peptide linker is from about 5 to about 30 amino acid residues in length. In embodiments, the peptide linker is from about 5 to about 25 amino acid residues in length. In embodiments, the peptide linker is from about 5 to about 20 amino acid residues in length. In embodiments, the peptide linker is from about 5 to about 15 amino acid residues in length. In embodiments, the peptide linker is from about 5 to about 10 amino acid residues in length. In embodiments, the peptide linker is 5, 10, 15, 20, 25, 30, 35, 40, 45, 50, 55, 60, 65, 70, 75, or 80 residues in length.

In embodiments, the peptide linker is from about 8 to about 30 amino acid residues in length. In embodiments, the peptide linker is from about 10 to about 30 amino acid residues in length. In embodiments, the peptide linker is from about 12 to about 30 amino acid residues in length. In embodiments, the peptide linker is from about 14 to about 30 amino acid residues in length. In embodiments, the peptide linker is from about 16 to about 30 amino acid residues in length. In embodiments, the peptide linker is from about 18 to about 30 amino acid residues in length. In embodiments, the peptide linker is from about 20 to about 30 amino acid residues in length. In embodiments, the peptide linker is from about 22 to about 30 amino acid residues in length. In embodiments, the peptide linker is from about 24 to about 30 amino acid residues in length. In embodiments, the peptide linker is from about 26 to about 30 amino acid residues in length. In embodiments, the peptide linker is from about 28 to about 30 amino acid residues in length.

In embodiments, the peptide linker is from about 8 to about 28 amino acid residues in length. In embodiments, the peptide linker is from about 8 to about 26 amino acid residues in length. In embodiments, the peptide linker is from about 8 to about 24 amino acid residues in length. In embodiments, the peptide linker is from about 8 to about 22 amino acid residues in length. In embodiments, the peptide linker is from about 8 to about 20 amino acid residues in length. In embodiments, the peptide linker is from about 8 to about 18 amino acid residues in length. In embodiments, the peptide linker is from about 8 to about 16 amino acid residues in length. In embodiments, the peptide linker is from about 8 to about 14 amino acid residues in length. In embodiments, the peptide linker is from about 8 to about 12 amino acid residues in length. In embodiments, the peptide linker is from about 8 to about 10 amino acid residues in length. In embodiments, the peptide linker is 8, 10, 12, 14, 16, 18, 20, 22, 24, 26, 28 or 30 amino acid residues in length.

In embodiments, the trimerization domain is a protein having a molecular weight of about 2 kDa to about 50 kDa. In embodiments, the trimerization domain is a protein having a molecular weight of about 4 kDa to about 50 kDa. In embodiments, the trimerization domain is a protein having a molecular weight of about 6 kDa to about 50 kDa. In embodiments, the trimerization domain is a protein having a molecular weight of about 8 kDa to about 50 kDa. In embodiments, the trimerization domain is a protein having a molecular weight of about 10 kDa to about 50 kDa. In embodiments, the trimerization domain is a protein having a molecular weight of about 12 kDa to about 50 kDa. In embodiments, the trimerization domain is a protein having a molecular weight of about 14 kDa to about 50 kDa. In embodiments, the trimerization domain is a protein having a molecular weight of about 16 kDa to about 50 kDa. In embodiments, the trimerization domain is a protein having a molecular weight of about 18 kDa to about 50 kDa. In embodiments, the trimerization domain is a protein having a molecular weight of about 20 kDa to about 50 kDa. In embodiments, the trimerization domain is a protein having a molecular weight of about 22 kDa to about 50 kDa. In embodiments, the trimerization domain is a protein having a molecular weight of about 24 kDa to about 50 kDa. In embodiments, the trimerization domain is a protein having a molecular weight of about 26 kDa to about 50 kDa. In embodiments, the trimerization domain is a protein having a molecular weight of about 28 kDa to about 50 kDa. In embodiments, the trimerization domain is a protein having a molecular weight of about 30 kDa to about 50 kDa. In embodiments, the trimerization domain is a protein having a molecular weight of about 32 kDa to about 50 kDa. In embodiments, the trimerization domain is a protein having a molecular weight of about 34 kDa to about 50 kDa. In embodiments, the trimerization domain is a protein having a molecular weight of about 36 kDa to about 50 kDa. In embodiments, the trimerization domain is a protein having a molecular weight of about 38 kDa to about 50 kDa. In embodiments, the trimerization domain is a protein having a molecular weight of about 40 kDa to about 50 kDa. In embodiments, the trimerization domain is a protein having a molecular weight of about 42 kDa to about 50 kDa. In embodiments, the trimerization domain is a protein having a molecular weight of about 44 kDa to about 50 kDa. In embodiments, the trimerization domain is a protein having a molecular weight of about 46 kDa to about 50 kDa. In embodiments, the trimerization domain is a protein having a molecular weight of about 48 kDa to about 50 kDa.

In embodiments, the trimerization domain is a protein having a molecular weight of about 2 kDa to about 48 kDa. In embodiments, the trimerization domain is a protein having a molecular weight of about 2 kDa to about 46 kDa. In embodiments, the trimerization domain is a protein having a molecular weight of about 2 kDa to about 44 kDa. In embodiments, the trimerization domain is a protein having a molecular weight of about 2 kDa to about 42 kDa. In embodiments, the trimerization domain is a protein having a molecular weight of about 2 kDa to about 40 kDa. In embodiments, the trimerization domain is a protein having a molecular weight of about 2 kDa to about 38 kDa. In embodiments, the trimerization domain is a protein having a molecular weight of about 2 kDa to about 36 kDa. In embodiments, the trimerization domain is a protein having a molecular weight of about 2 kDa to about 34 kDa. In embodiments, the trimerization domain is a protein having a molecular weight of about 2 kDa to about 32 kDa. In embodiments, the trimerization domain is a protein having a molecular weight of about 2 kDa to about 30 kDa. In embodiments, the trimerization domain is a protein having a molecular weight of about 2 kDa to about 28 kDa. In embodiments, the trimerization domain is a protein having a molecular weight of about 2 kDa to about 26 kDa. In embodiments, the trimerization domain is a protein having a molecular weight of about 2 kDa to about 24 kDa. In embodiments, the trimerization domain is a protein having a molecular weight of about 2 kDa to about 22 kDa. In embodiments, the trimerization domain is a protein having a molecular weight of about 2 kDa to about 20 kDa. In embodiments, the trimerization domain is a protein having a molecular weight of about 2 kDa to about 18 kDa. In embodiments, the trimerization domain is a protein having a molecular weight of about 2 kDa to about 16 kDa. In embodiments, the trimerization domain is a protein having a molecular weight of about 2 kDa to about 14 kDa. In embodiments, the trimerization domain is a protein having a molecular weight of about 2 kDa to about 12 kDa. In embodiments, the trimerization domain is a protein having a molecular weight of about 2 kDa to about 10 kDa. In embodiments, the trimerization domain is a protein having a molecular weight of about 2 kDa to about 8 kDa. In embodiments, the trimerization domain is a protein having a molecular weight of about 2 kDa to about 6 kDa. In embodiments, the trimerization domain is a protein having a molecular weight of about 2 kDa to about 4 kDa.

In embodiments, the trimerization domain is a protein having a molecular weight of 2 kDa, 4 kDa, 6 kDa, 8 kDa, 10 kDa, 12 kDa, 14 kDa, 16 kDa, 18 kDa, 20 kDa, 22 kDa, 24 kDa, 26 kDa, 28 kDa, 30 kDa, 32 kDa, 34 kDa, 36 kDa, 38 kDa, 40 kDa, 42 kDa, 44 kDa, 46 kDa, 48 kDa, or 50 kDa.

In embodiments, the trimerization domain is a protein having a molecular weight of about 3 kDa to about 30 kDa. In embodiments, the trimerization domain is a protein having a molecular weight of about 6 kDa to about 30 kDa. In embodiments, the trimerization domain is a protein having a molecular weight of about 9 kDa to about 30 kDa. In embodiments, the trimerization domain is a protein having a molecular weight of about 12 kDa to about 30 kDa. In embodiments, the trimerization domain is a protein having a molecular weight of about 15 kDa to about 30 kDa. In embodiments, the trimerization domain is a protein having a molecular weight of about 18 kDa to about 30 kDa. In embodiments, the trimerization domain is a protein having a molecular weight of about 21 kDa to about 30 kDa. In embodiments, the trimerization domain is a protein having a molecular weight of about 24 kDa to about 30 kDa. In embodiments, the trimerization domain is a protein having a molecular weight of about 27 kDa to about 30 kDa.

In embodiments, the trimerization domain is a protein having a molecular weight of about 3 kDa to about 27 kDa. In embodiments, the trimerization domain is a protein having a molecular weight of about 3 kDa to about 24 kDa. In embodiments, the trimerization domain is a protein having a molecular weight of about 3 kDa to about 21 kDa. In embodiments, the trimerization domain is a protein having a molecular weight of about 3 kDa to about 18 kDa. In embodiments, the trimerization domain is a protein having a molecular weight of about 3 kDa to about 15 kDa. In embodiments, the trimerization domain is a protein having a molecular weight of about 3 kDa to about 12 kDa. In embodiments, the trimerization domain is a protein having a molecular weight of about 3 kDa to about 8 kDa. In embodiments, the trimerization domain is a protein having a molecular weight of about 3 kDa to about 6 kDa. In embodiments, the trimerization domain is a protein having a molecular weight of 3 kDa, 6 kDa, 9 kDa, 12 kDa, 15 kDa, 18 kDa, 21 kDa, 24 kDa, 27 kDa, or 30 kDa.

The ability of a monomer to bind its component monomers to form a trimer can be described can be described by the equilibrium dissociation constant (K_{D}). The equilibrium dissociation constant (K_{D}) as defined herein is the ratio of the dissociation rate (K-off) and the association rate (K-on) of a monomer to the component monomers of the trimer. It is described by the following formula: K_{D}=K-off/K-on.

In embodiments, a monomer of the trimer binds a component monomer with an equilibrium dissociation constant (K_{D}) from 0.01 nM to 50 nM. In embodiments, a monomer of the trimer binds a component monomer with a K_{D} from 1 nM to 50 nM. In embodiments, a monomer of the trimer binds a component monomer with a K_{D} from 5 nM to 50 nM. In embodiments, a monomer of the trimer binds a component monomer with a K_{D} from 10 nM to 50 nM. In embodiments, a monomer of the trimer binds a component monomer with a K_{D} from 15 nM to 50 nM. In embodiments, a monomer of the trimer binds a component monomer with a K_{D} from 20 nM to 50 nM. In embodiments, a monomer of the trimer binds a component monomer with a K_{D} from 25 nM to 50 nM. In embodiments, a monomer of the trimer binds a component monomer with a K_{D} from 30 nM to 50 nM. In embodiments, a monomer of the trimer binds a component monomer with a K_{D} from 35 nM to 50 nM. In embodiments, a monomer of the trimer binds a component monomer with a K_{D} from 40 nM to 50 nM. In embodiments, a monomer of the trimer binds a component monomer with a K_{D} from 45 nM to 50 nM.

In embodiments, a monomer of the trimer binds a component monomer with a K_{D} from 0.01 nM to 45 nM. In embodiments, a monomer of the trimer binds a component monomer with a K_{D} from 0.01 nM to 40 nM. In embodiments, a monomer of the trimer binds a component monomer with a K_{D} from 0.01 nM to 35 nM. In embodiments, a monomer of the trimer binds a component monomer with a K_{D} from 0.01 nM to 30 nM. In embodiments, a monomer of the trimer binds a component monomer with a K_{D} from 0.01 nM to 25 nM. In embodiments, a monomer of the trimer binds a component monomer with a K_{D} from 0.01 nM to 20 nM. In embodiments, a monomer of the trimer binds a component monomer with a K_{D} from 0.01 nM to 15 nM. In embodiments, a monomer of the trimer binds a component monomer with a K_{D} from 0.01 nM to 10 nM. In embodiments, a monomer of the trimer binds a component monomer with a K_{D} from 0.01 nM to 5 nM. In embodiments, a monomer of the trimer binds a component monomer with a K_{D} from 0.01 nM to 1 nM. In embodiments, a monomer of the trimer binds a component monomer with a K_{D} of 0.01 nM, 1 nM, 5 nM, 10 nM, 15 nM, 20 nM, 25 nM, 30 nM, 35 nM, 40 nM, 45 nM, or 50 nM.

In embodiments, a monomer of the trimer binds a component monomer with an equilibrium dissociation constant (K_{D}) from 0.01 nM to 20 nM. In embodiments, a monomer of the trimer binds a component monomer with an equilibrium dissociation constant (K_{D}) from 0.5 nM to 20 nM. In embodiments, a monomer of the trimer binds a component monomer with an equilibrium dissociation constant (K_{D}) from 1 nM to 20 nM. In embodiments, a monomer of the trimer binds a component monomer with an equilibrium dissociation constant (K_{D}) from 1.5 nM to 20 nM. In embodiments, a monomer of the trimer binds a component monomer with an equilibrium dissociation constant (K_{D}) from 2 nM to 20 nM. In embodiments, a monomer of the trimer binds a component monomer with an equilibrium dissociation constant (K_{D}) from 2.5 nM to 20 nM. In embodiments, a monomer of the trimer binds a component monomer with an equilibrium dissociation constant (K_{D}) from 3 nM to 20 nM. In embodiments, a monomer of the trimer binds a component monomer with an equilibrium dissociation constant (K_{D}) from 3.5 nM to 20 nM. In embodiments, a monomer of the trimer binds a component monomer with an equilibrium dissociation constant (K_{D}) from 4 nM to 20 nM. In embodiments, a monomer of the trimer binds a component monomer with an equilibrium dissociation constant (K_{D}) from 4.5 nM to 20 nM. In embodiments, a monomer of the trimer binds a component monomer with an equilibrium dissociation constant (K_{D}) from 5 nM to 20 nM. In embodiments, a monomer of the trimer binds a component monomer with an equilibrium dissociation constant (K_{D}) from 5.5 nM to 20 nM. In embodiments, a monomer of the trimer binds a component monomer with an equilibrium dissociation constant (K_{D}) from 6 nM to 20 nM. In embodiments, a monomer of the trimer binds a component monomer with an equilibrium dissociation constant (K_{D}) from 6.5 nM to 20 nM. In embodiments, a monomer of the trimer binds a component monomer with an equilibrium dissociation constant (K_{D}) from 7 nM to 20 nM. In embodiments, a monomer of the trimer binds a component monomer with an equilibrium dissociation constant (K_{D}) from 7.5 nM to 20 nM. In embodiments, a monomer of the trimer binds a component monomer with an equilibrium dissociation constant (K_{D}) from 8 nM to 20 nM. In embodiments, a monomer of the trimer binds a component monomer with an equilibrium dissociation constant (K_{D}) from 8.5 nM to 20 nM. In embodiments, a monomer of the trimer binds a component monomer with an equilibrium dissociation constant (K_{D}) from 9 nM to 20 nM. In embodiments, a monomer of the trimer binds a component monomer with an equilibrium dissociation constant (K_{D}) from 9.5 nM to 20 nM. In embodiments, a monomer of the trimer binds a component monomer with an equilibrium dissociation constant (K_{D}) from 10 nM to 20 nM. In embodiments, a monomer of the trimer binds a component monomer with an equilibrium dissociation constant (K_{D}) from 10.5 nM to 20 nM. In embodiments, a monomer of the trimer binds a component monomer with an equilibrium dissociation constant (K_{D}) from 11 nM to 20 nM. In embodiments, a monomer of the trimer binds a component monomer with an equilibrium dissociation constant (K_{D}) from 11.5 nM to 20 nM. In embodiments, a monomer of the trimer binds a component monomer with an equilibrium dissociation constant (K_{D}) from 12 nM to 20 nM. In embodiments, a monomer of the trimer binds a component monomer with an equilibrium dissociation constant (K_{D}) from 12.5 nM to 20 nM. In embodiments, a monomer of the trimer binds a component monomer with an equilibrium dissociation constant (K_{D}) from 13 nM to 20 nM. In embodiments, a monomer of the trimer binds a component monomer with an equilibrium dissociation constant (K_{D}) from 13.5 nM to 20 nM. In embodiments, a monomer of the trimer binds a component monomer with an equilibrium dissociation constant (K_{D}) from 14 nM to 20 nM. In embodiments, a monomer of the trimer binds a component monomer with an equilibrium dissociation constant (K_{D}) from 14.5 nM to 20 nM. In embodiments, a monomer of the trimer binds a component monomer with an equilibrium dissociation constant (K_{D}) from 15 nM to 20 nM. In embodiments, a monomer of the trimer binds a component monomer with an equilibrium dissociation constant (K_{D}) from 15.5 nM to 20 nM. In embodiments, a monomer of the trimer binds a component monomer with an equilibrium dissociation constant (K_{D}) from 16 nM to 20 nM. In embodiments, a monomer of the trimer binds a component monomer with an equilibrium dissociation constant (K_{D}) from 16.5 nM to 20 nM. In embodiments, a monomer of the trimer binds a component monomer with an equilibrium dissociation constant (K_{D}) from 17 nM to 20 nM. In embodiments, a monomer of the trimer binds a component monomer with an equilibrium dissociation constant (K_{D}) from 17.5 nM to 20 nM. In embodiments, a monomer of the trimer binds a component monomer with an equilibrium dissociation constant (K_{D}) from 18 nM to 20 nM. In embodiments, a monomer of the trimer binds a component monomer with an equilibrium dissociation constant (K_{D}) from 18.5 nM to 20 nM. In embodiments, a monomer of the trimer binds a component monomer with an equilibrium dissociation constant (K_{D}) from 19 nM to 20 nM. In embodiments, a monomer of the trimer binds a component monomer with an equilibrium dissociation constant (K_{D}) from 19.5 nM to 20 nM.

In embodiments, a monomer of the trimer binds a component monomer with an equilibrium dissociation constant (K_{D}) from 0.01 nM to 19.5 nM. In embodiments, a monomer of the trimer binds a component monomer with an equilibrium dissociation constant (K_{D}) from 0.01 nM to 19 nM. In embodiments, a monomer of the trimer binds a component monomer with an equilibrium dissociation constant (K_{D}) from 0.01 nM to 18.5 nM. In embodiments, a monomer of the trimer binds a component monomer with an equilibrium dissociation constant (K_{D}) from 0.01 nM to 18 nM. In embodiments, a monomer of the trimer binds a component monomer with an equilibrium dissociation constant (K_{D}) from 0.01 nM to 17.5 nM. In embodiments, a monomer of the trimer binds a component monomer with an equilibrium dissociation constant (K_{D}) from 0.01 nM to 17 nM. In embodiments, a monomer of the trimer binds a component monomer with an equilibrium dissociation constant (K_{D}) from 0.01 nM to 16.5 nM. In embodiments, a monomer of the trimer binds a component monomer with an equilibrium dissociation constant (K_{D}) from 0.01 nM to 16 nM. In embodiments, a monomer of the trimer binds a component monomer with an equilibrium dissociation constant (K_{D}) from 0.01 nM to 15.5 nM. In embodiments, a monomer of the trimer binds a component monomer with an equilibrium dissociation constant (K_{D}) from 0.01 nM to 15 nM. In embodiments, a monomer of the trimer binds a component monomer with an equilibrium dissociation constant (K_{D}) from 0.01 nM to 14.5 nM. In embodiments, a monomer of the trimer binds a component monomer with an equilibrium dissociation constant (K_{D}) from 0.01 nM to 14 nM. In embodiments, a monomer of the trimer binds a component monomer with an equilibrium dissociation constant (K_{D}) from 0.01 nM to 13.5 nM. In embodiments, a monomer of the trimer binds a component monomer with an equilibrium dissociation constant (K_{D}) from 0.01 nM to 13 nM. In embodiments, a monomer of the trimer binds a component monomer with an equilibrium dissociation constant (K_{D}) from 0.01 nM to 12.5 nM. In embodiments, a monomer of the trimer binds a component monomer with an equilibrium dissociation constant (K_{D}) from 0.01 nM to 12 nM. In embodiments, a monomer of the trimer binds a component monomer with an equilibrium dissociation constant (K_{D}) from 0.01 nM to 11.5 nM. In embodiments, a monomer of the trimer binds a component monomer with an equilibrium dissociation constant (K_{D}) from 0.01 nM to 11 nM. In embodiments, a monomer of the trimer binds a component monomer with an equilibrium dissociation constant (K_{D}) from 0.01 nM to 10.5 nM. In embodiments, a monomer of the trimer binds a component monomer with an equilibrium dissociation constant (K_{D}) from 0.01 nM to 10 nM. In embodiments, a monomer of the trimer binds a component monomer with an equilibrium dissociation constant (K_{D}) from 0.01 nM to 9.5 nM. In embodiments, a monomer of the trimer binds a component monomer with an equilibrium dissociation constant (K_{D}) from 0.01 nM to 9 nM. In embodiments, a monomer of the trimer binds a component monomer with an equilibrium dissociation constant (K_{D}) from 0.01 nM to 8.5 nM. In embodiments, a monomer of the trimer binds a component monomer with an equilibrium dissociation constant (K_{D}) from 0.01 nM to 8 nM. In embodiments, a monomer of the trimer binds a component monomer with an equilibrium dissociation constant (K_{D}) from 0.01 nM to 7.5 nM. In embodiments, a monomer of the trimer binds a component monomer with an equilibrium dissociation constant (K_{D}) from 0.01 nM to 7 nM. In embodiments, a monomer of the trimer binds a component monomer with an equilibrium dissociation constant (K_{D}) from 0.01 nM to 6.5 nM. In embodiments, a monomer of the trimer binds a component monomer with an equilibrium dissociation constant (K_{D}) from 0.01 nM to 6 nM. In embodiments, a monomer of the trimer binds a component monomer with an equilibrium dissociation constant (K_{D}) from 0.01 nM to 5.5 nM. In embodiments, a monomer of the trimer binds a component monomer with an equilibrium dissociation constant (K_{D}) from 0.01 nM to 5 nM. In embodiments, a monomer of the trimer binds a component monomer with an equilibrium dissociation constant (K_{D}) from 0.01 nM to 4.5 nM. In embodiments, a monomer of the trimer binds a component monomer with an equilibrium dissociation constant (K_{D}) from 0.01 nM to 4 nM. In embodiments, a monomer of the trimer binds a component monomer with an equilibrium dissociation constant (K_{D}) from 0.01 nM to 3.5 nM. In embodiments, a monomer of the trimer binds a component monomer with an equilibrium dissociation constant (K_{D}) from 0.01 nM to 3 nM. In embodiments, a monomer of the trimer binds a component monomer with an equilibrium dissociation constant (K_{D}) from 0.01 nM to 2.5 nM. In embodiments, a monomer of the trimer binds a component monomer with an equilibrium dissociation constant (K_{D}) from 0.01 nM to 2 nM. In embodiments, a monomer of the trimer binds a component monomer with an equilibrium dissociation constant (K_{D}) from 0.01 nM to 1.5 nM. In embodiments, a monomer of the trimer binds a component monomer with an equilibrium dissociation constant (K_{D}) from 0.01 nM to 1 nM. In embodiments, a monomer of the trimer binds a component monomer with an equilibrium dissociation constant (K_{D}) from 0.01 nM to 0.5 nM. In embodiments, a monomer of the trimer binds a component monomer with an equilibrium dissociation constant (K_{D}) of 0.01 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, or 20 nM.

In embodiments, the trimerization domain is a leucine zipper, 1NOG, Fibritin, or GCN4. In embodiments, the trimerization domain is a leucine zipper domain. In embodiments, the trimerization domain is 1NOG. In embodiments, the trimerization domain is Fibritin. In embodiments, the trimerization domain is GCN4. The trimerization domain may be any protein domain that is capable of forming a trimer. Trimerization domains suitable for use are provided and discussed in greater detail in Morris, C. D. et al. Differential Antibody Responses to Conserved HIV-1 Neutralizing Epitopes in the Context of Multivalent Scaffolds and Native-Like gp140 Trimers. mBio, January/February 2017 Volume 8 Issue 1 e00036-17.; https://doi.org/10.1128/mBio.00036-17.; which is incorporated herein by reference in its entirety for all purposes.

In embodiments, the trimerization domain is the amino acid sequence of SEQ ID NO:5. In embodiments, the trimerization domain includes the amino acid sequence of SEQ ID NO:5. In embodiments, the trimerization domain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:5. In embodiments, the trimerization domain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:5.

In embodiments, the trimerization domain is the amino acid sequence of SEQ ID NO:11. In embodiments, the trimerization domain includes the amino acid sequence of SEQ ID NO:11. In embodiments, the trimerization domain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:11. In embodiments, the trimerization domain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:11.

For the recombinant arenavirus glycoprotein provided herein including embodiments thereof, in embodiments, the ectodomain of the arenavirus glycoprotein polypeptide includes: an arginine at a position corresponding to position 258 of SEQ ID NO:18, an arginine at a position corresponding to position 259 of SEQ ID NO:18, and a proline at a position corresponding to position 329 of SEQ ID NO:18. In embodiments, the ectodomain includes: a phenylalanine at a position corresponding to position 193 of SEQ ID NO:18, a leucine at a position corresponding to position 211 of SEQ ID NO:18, a leucine at a position corresponding to position 339 of SEQ ID NO:18, and a leucine at a position corresponding to position 354 of SEQ ID NO:18.

In embodiments, the ectodomain further includes: a proline at a position corresponding to position 128 of SEQ ID NO:18, or an isoleucine, leucine or valine at a position corresponding to position 305 of SEQ ID NO:18. In embodiments, the ectodomain further includes: a proline at a position corresponding to position 128 of SEQ ID NO:18. In embodiments, the ectodomain further includes: an isoleucine at a position corresponding to position 305 of SEQ ID NO:18. In embodiments, the ectodomain further includes: a leucine at a position corresponding to position 305 of SEQ ID NO:18. In embodiments, the ectodomain further includes: a valine at a position corresponding to position 305 of SEQ ID NO:18.

In embodiments, the ectodomain includes an amino acid sequence having a sequence identify of at least 80% to the amino acid sequence of SEQ ID NO:27. In embodiments, the ectodomain includes an amino acid sequence having a sequence identify of at least 85% to the amino acid sequence of SEQ ID NO:27. In embodiments, the ectodomain includes an amino acid sequence having a sequence identify of at least 90% to the amino acid sequence of SEQ ID NO:27. In embodiments, the ectodomain includes an amino acid sequence having a sequence identify of at least 95% to the amino acid sequence of SEQ ID NO:27. In embodiments, the ectodomain includes an amino acid sequence having a sequence identify of at least 96% to the amino acid sequence of SEQ ID NO:27. In embodiments, the ectodomain includes an amino acid sequence having a sequence identify of at least 97% to the amino acid sequence of SEQ ID NO:27. In embodiments, the ectodomain includes an amino acid sequence having a sequence identify of at least 98% to the amino acid sequence of SEQ ID NO:27. In embodiments, the ectodomain includes an amino acid sequence having a sequence identify of at least 99% to the amino acid sequence of SEQ ID NO:27. In embodiments, the ectodomain includes the amino acid sequence of SEQ ID NO:27. In embodiments, the ectodomain is the amino acid sequence of SEQ ID NO:27.

For the recombinant arenavirus glycoprotein provided herein including embodiments thereof, in embodiments, the ectodomain further includes a signal peptide. In embodiments, the signal peptide is covalently bound to the N-terminus of the GP1 domain. In embodiments, the C-terminus of the signal peptide is covalently bound to the N-terminus of the GP1 domain. For example, the signal peptide may be bound to the GP1 domain by way of a covalent bond (e.g. a peptide bond). In embodiments, the signal peptide is attached to the GP1 domain by a peptide linker.

In embodiments, the signal peptide is the amino acid sequence of SEQ ID NO:2. In embodiments, the signal peptide includes the amino acid sequence of SEQ ID NO:2. In embodiments, the signal peptide is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:2. In embodiments, the signal peptide is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:2.

In instances, glycosylation or lack thereof at specific residues of the arenavirus glycoprotein ectodomain improves recognition of an anti-glycoprotein antibody to the ectodomain. The term “glycosylation” as provided herein is used according to its common meaning in the art and refers to attachment of a carbohydrate (e.g. glycan) to an amino acid side chain within a protein or polypeptide. In embodiments, glycosylation is N-linked, wherein a glycan is attached to a nitrogen atom of an Asp or Arg side chain. In embodiments, glycosylation is O-linked, wherein a glycan is attached to a hydroxyl oxygen of an amino acid side chain (e.g. Ser, Thr, Tyr, hydroxylysine, hydroxyproline, etc.).

Thus, for the recombinant arenavirus glycoprotein provided herein, in embodiments, the ectodomain is non-glycosylated. In embodiments, the ectodomain is glycosylated. In embodiments, the ectodomain is glycosylated at one or more amino acid residues at positions corresponding to 79, 89, 99, 119, 365 and 373 of SEQ ID NO:18. In embodiments, the ectodomain is glycosylated at an amino acid residue corresponding to position 79 of SEQ ID NO:18. In embodiments, the ectodomain is glycosylated at an amino acid residue corresponding to position 89 of SEQ ID NO:18. In embodiments, the ectodomain is glycosylated at an amino acid residue corresponding to position 99 of SEQ ID NO:18. In embodiments, the ectodomain is glycosylated at an amino acid residue corresponding to position 119 of SEQ ID NO:18. In embodiments, the ectodomain is glycosylated at an amino acid residue corresponding to position 365 of SEQ ID NO:18. In embodiments, the ectodomain is glycosylated at an amino acid residue corresponding to position 373 of SEQ ID NO:18. In embodiments, the ectodomain is not glycosylated at one or more amino acid residues at positions corresponding to 390 or 395 of SEQ ID NO:18. In embodiments, the ectodomain is not glycosylated at a position corresponding to 390 of SEQ ID NO:18. In embodiments, the ectodomain is not glycosylated at a position corresponding to 395 of SEQ ID NO:18.

In embodiments, the arenavirus glycoprotein ectodomain includes the amino acid sequence of SEQ ID NO:1. In embodiments, the arenavirus glycoprotein ectodomain is the amino acid sequence of SEQ ID NO:1. In embodiments, the arenavirus glycoprotein ectodomain is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:1. In embodiments, the arenavirus glycoprotein ectodomain is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:1.

The recombinant arenavirus glycoprotein provided herein including embodiments thereof is capable of forming a glycoprotein trimer. Applicant has discovered that neutralizing antibodies may recognize and bind the glycoprotein trimer. Thus, in an aspect is provided a glycoprotein trimer, wherein the trimer includes three of the recombinant arenavirus glycoproteins provided herein including embodiments thereof, wherein the three recombinant arenavirus glycoproteins are bound by non-covalent attachment of the trimerization domains.

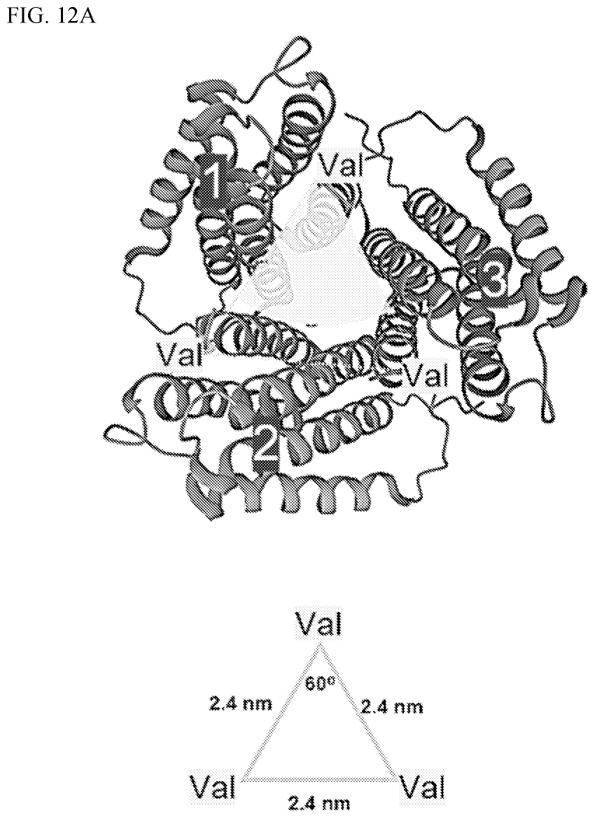

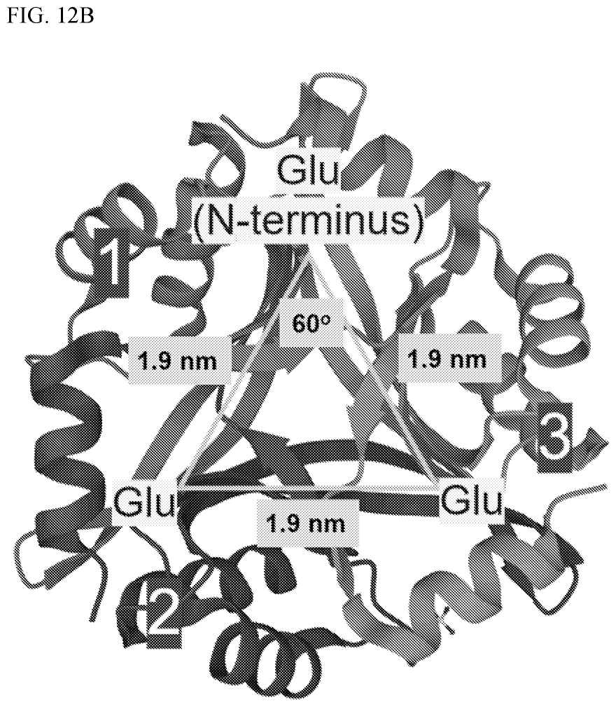

In embodiments, the N-termini of the trimerization domains may be positioned towards the perimeter of the trimer. Thus, in embodiments, the N-termini of the trimerization domains are positioned towards the outer edges of the trimer rather than the center of the trimeric axis. In embodiments, the N-termini of the trimerization domains are positioned towards the outer edges of the trimer rather than the center of the trimeric axis, as shown in FIG. 12B. Thus, in embodiments, a first monomer of the trimerization domain is oriented 30-100° from the second monomer and third monomer of the trimerization domain. In embodiments, a first monomer of the trimerization domain is oriented 40-100° from the second monomer and third monomer of the trimerization domain. In embodiments, a first monomer of the trimerization domain is oriented 50-100° from the second monomer and third monomer of the trimerization domain. In embodiments, a first monomer of the trimerization domain is oriented 60-100° from the second monomer and third monomer of the trimerization domain. In embodiments, a first monomer of the trimerization domain is oriented 70-100° from the second monomer and third monomer of the trimerization domain. In embodiments, a first monomer of the trimerization domain is oriented 80-100° from the second monomer and third monomer of the trimerization domain. In embodiments, a first monomer of the trimerization domain is oriented 90-100° from the second monomer and third monomer of the trimerization domain.

In embodiments, a first monomer of the trimerization domain is oriented 30-90° from the second monomer and third monomer of the trimerization domain. In embodiments, a first monomer of the trimerization domain is oriented 30-80° from the second monomer and third monomer of the trimerization domain. In embodiments, a first monomer of the trimerization domain is oriented 30-70° from the second monomer and third monomer of the trimerization domain. In embodiments, a first monomer of the trimerization domain is oriented 30-60° from the second monomer and third monomer of the trimerization domain. In embodiments, a first monomer of the trimerization domain is oriented 30-50° from the second monomer and third monomer of the trimerization domain. In embodiments, a first monomer of the trimerization domain is oriented 30-40° from the second monomer and third monomer of the trimerization domain. In embodiments, a first monomer of the trimerization domain is oriented 30°, 40°, 50°, 60°, 70°, 80° or 90°, from the second monomer and third monomer of the trimerization domain. In embodiments, a first monomer of the trimerization domain is oriented 60° from the second monomer and third monomer of the trimerization domain.

In embodiments, a first monomer of the trimerization domain is equidistant from the second monomer and third monomer of the trimerization domain. In embodiments, a first monomer of the trimerization domain is 0.5 nm to 4 nm in distance from the second monomer and third monomer of the trimerization domain. In embodiments, a first monomer of the trimerization domain is 0.75 nm to 4 nm in distance from the second monomer and third monomer of the trimerization domain. In embodiments, a first monomer of the trimerization domain is 1 nm to 4 nm in distance from the second monomer and third monomer of the trimerization domain. In embodiments, a first monomer of the trimerization domain is 1.25 nm to 4 nm in distance from the second monomer and third monomer of the trimerization domain. In embodiments, a first monomer of the trimerization domain is 1.5 nm to 4 nm in distance from the second monomer and third monomer of the trimerization domain. In embodiments, a first monomer of the trimerization domain is 1.75 nm to 4 nm in distance from the second monomer and third monomer of the trimerization domain. In embodiments, a first monomer of the trimerization domain is 2 nm to 4 nm in distance from the second monomer and third monomer of the trimerization domain. In embodiments, a first monomer of the trimerization domain is 2.25 nm to 4 nm in distance from the second monomer and third monomer of the trimerization domain. In embodiments, a first monomer of the trimerization domain is 2.5 nm to about 4 nm in from the second monomer and third monomer of the trimerization domain. In embodiments, a first monomer of the trimerization domain is 2.75 nm to 4 nm in distance from the second monomer and third monomer of the trimerization domain. In embodiments, a first monomer of the trimerization domain is 3 nm to 4 nm in distance from the second monomer and third monomer of the trimerization domain. In embodiments, a first monomer of the trimerization domain is 3.25 nm to 4 nm in distance from the second monomer and third monomer of the trimerization domain. In embodiments, a first monomer of the trimerization domain is 3.5 nm to 4 nm in distance from the second monomer and third monomer of the trimerization domain. In embodiments, a first monomer of the trimerization domain is 3.75 nm to 4 nm in distance from the second monomer and third monomer of the trimerization domain.

In embodiments, a first monomer of the trimerization domain is 0.5 nm to 3.75 nm in distance from the second monomer and third monomer of the trimerization domain. In embodiments, a first monomer of the trimerization domain is 0.5 nm to 3.5 nm in distance from the second monomer and third monomer of the trimerization domain. In embodiments, a first monomer of the trimerization domain is 0.5 nm to 3.25 nm in distance from the second monomer and third monomer of the trimerization domain. In embodiments, a first monomer of the trimerization domain is 0.5 nm to 3 nm in distance from the second monomer and third monomer of the trimerization domain. In embodiments, a first monomer of the trimerization domain is 0.5 nm to 2.75 nm in distance from the second monomer and third monomer of the trimerization domain. In embodiments, a first monomer of the trimerization domain is 0.5 nm to 2.5 nm in distance from the second monomer and third monomer of the trimerization domain. In embodiments, a first monomer of the trimerization domain is 0.5 nm to 2.25 nm in distance from the second monomer and third monomer of the trimerization domain. In embodiments, a first monomer of the trimerization domain is 0.5 nm to 2 nm in distance from the second monomer and third monomer of the trimerization domain. In embodiments, a first monomer of the trimerization domain is 0.5 nm to 1.75 nm in distance from the second monomer and third monomer of the trimerization domain. In embodiments, a first monomer of the trimerization domain is 0.5 nm to 1.5 nm in distance from the second monomer and third monomer of the trimerization domain. In embodiments, a first monomer of the trimerization domain is 0.5 nm to 1.25 nm in distance from the second monomer and third monomer of the trimerization domain. In embodiments, a first monomer of the trimerization domain is 0.5 nm to 1 nm in distance from the second monomer and third monomer of the trimerization domain. In embodiments, a first monomer of the trimerization domain is 0.5 nm to 0.75 nm in distance from the second monomer and third monomer of the trimerization domain. In embodiments, a first monomer of the trimerization domain is 0.5 nm, 0.75 nm, 1 nm, 1.25 nm, 1.5 nm, 1.75 nm, 2 nm, 2.25 nm, 2.5 nm, 2.75 nm, 3 nm, 3.25 nm, 3.5 nm or 4 nm in distance from the second monomer and third monomer of the trimerization domain. In embodiments, a first monomer of the trimerization domain is 1.9 nm in distance from the second monomer and third monomer of the trimerization domain.

In an aspect is provided an antibody that binds a recombinant arenavirus glycoprotein provided herein including embodiments thereof. In an aspect is provided an antibody that binds a glycoprotein trimer provided herein including embodiments thereof.

### Nucleic Acid Compositions

In an aspect is provided a nucleic acid encoding the recombinant arenavirus glycoprotein provided herein including embodiments thereof. The nucleic acid provided herein, including embodiments thereof, may be loaded into an expression vector such that the nucleic acid may be delivered to cells. Thus, in an aspect, an expression vector including the nucleic acid provided herein, including embodiments thereof, is provided. It is contemplated that the nucleic acid may be loaded into any expression vector useful for delivering the nucleic acid to cells either in vivo or in vitro.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

### Cells

In an aspect is provided a cell including the recombinant arenavirus glycoprotein provided herein including embodiments thereof or the glycoprotein trimer provided herein including embodiments thereof. In another aspect is provided a cell including the nucleic acid provided herein including embodiments thereof. In embodiments, the cell is a human cell.

### Vaccine Compositions

The recombinant arenavirus glycoprotein and glycoprotein trimer provided herein including embodiments thereof are contemplated to be particularly effective in vaccine compositions for treating and/or preventing arenavirus infections. Applicant has found that the glycoprotein trimer is recognized by anti-glycoprotein antibodies, and may be a target for antibody-mediated neutralization of arenavirus infection. Thus, in an aspect is provided a vaccine composition including the recombinant arenavirus glycoprotein provided herein including embodiments thereof and a pharmaceutically acceptable excipient. In another aspect is provided a vaccine composition including the glycoprotein trimer provided herein including embodiments thereof and a pharmaceutically acceptable excipient.

In embodiments, the vaccine composition further includes one or more of a stabilizer, an adjuvant, and a preservative. In embodiments, the vaccine composition includes a stabilizer. In embodiments, the vaccine composition includes a preservative. In embodiments, the vaccine further comprises an adjuvant. In embodiments, the adjuvant is a gel-type, microbial, particulate, oil-emulsion, surfactant-based, or synthetic adjuvant. In embodiments, the adjuvant is aluminum hydroxide/phosphate, calcium phosphate, muramyl dipeptide (MDP), a bacterial exotoxin, an endotoxin-based adjuvant, a biodegradable adjuvant, polymer microspheres, immunostimulatory complexes (ISCOMs), liposomes, Freund's incomplete adjuvant, microfluidized emulsions, saponins, muramyl peptide derivatives, nonionic block copolymers, polyphosphazene (PCPP), synthetic polynucleotide, or a thalidomide derivative. In embodiments, the adjuvant is a CpG oligonucleotide. In embodiments, the adjuvant comprises a BCG sequence. In embodiments, the adjuvant is a tetanus toxoid.

### Methods

The recombinant arenavirus glycoprotein provided herein including embodiments thereof is particularly useful for treating or preventing arenavirus infections. Thus, in an aspect is provided a method of treating or preventing a viral disease in a subject in need of such treatment or prevention, the method including administering a therapeutically or prophylactically effective amount of the recombinant arenavirus glycoprotein provided herein including embodiments thereof to the subject. In another aspect is provided a method of treating or preventing a viral disease in a subject in need of such treatment or prevention, the method including administering a therapeutically or prophylactically effective amount of the glycoprotein trimer provided herein including embodiments thereof to the subject.

In embodiments, the viral disease is caused by Lymphocytic choriomeningitis virus (LCMV), Junin virus, Machupo virus, Lassa virus, Guanarito virus, Sabia virus, Chapare virus, Whitewater Arroyo virus, or Lujo virus. In embodiments, the viral disease is caused by LCMV. In embodiments, the viral disease is caused by Junin virus. In embodiments, the viral disease is caused by Machupo virus. In embodiments, the viral disease is caused by Lassa virus. In embodiments, the viral disease is caused by Guanarito virus. In embodiments, the viral disease is caused by Sabia virus. In embodiments, the viral disease is caused by Chapare virus. In embodiments, the viral disease is caused by Whitewater Arroyo virus. In embodiments, the viral disease is caused by Lujo virus.

In an aspect is provided a method for immunizing a subject susceptible to a viral disease, the method including administering the recombinant arenavirus glycoprotein provided herein including embodiments thereof to a subject under conditions such that antibodies directed to the arenavirus glycoprotein or a fragment thereof are produced. In an aspect is provided a method for immunizing a subject susceptible to a viral disease, the method including administering the glycoprotein trimer provided herein including embodiments thereof to a subject under conditions such that antibodies directed to the glycoprotein trimer or a fragment thereof are produced.

The recombinant arenavirus glycoproteins and glycoprotein trimers provided herein including embodiments thereof are contemplated to be useful for as research tools in a variety of clinical or research applications. These include, e.g., characterizing the desired types of antibodies in a discovery effort for immunotherapeutics or diagnostics, and characterizing the desired types of antibody responses elicited by a vaccine or a natural infection. Thus, in an aspect is provided a method of diagnosing arenavirus infection in a subject, the method including: (a) contacting a biological sample obtained from the subject with the recombinant arenavirus glycoprotein provided herein including embodiments thereof, and (b) detecting binding of one or more antibodies to the recombinant arenavirus glycoprotein, thereby diagnosing arenavirus infection in the subject. In another aspect is provided a method of diagnosing arenavirus infection in a subject, the method including: (a) contacting a biological sample obtained from the subject with the glycoprotein trimer provided herein including embodiments thereof, and (b) detecting binding of one or more antibodies to the glycoprotein trimer, thereby diagnosing arenavirus infection in the subject.

In embodiments, the recombinant arenavirus glycoproteins and glycoprotein trimers are used to diagnose arenaviral infections (e.g., LASV infections). In embodiments, a biological sample is typically obtained from subjects suspected of having an arenaviral infection. The biological sample may be any tissue or liquid sample from the subject. In embodiments, the sample is a blood sample, e.g., whole blood, plasma or serum. The sample is then contacted with an recombinant arenavirus glycoprotein or glycoprotein trimer provided herein including embodiments thereof to allow detection of both neutralizing and non-neutralizing antibodies against arenaviral GP. In embodiments, the recombinant arenavirus glycoproteins and glycoprotein trimers provided herein including embodiments thereof can be employed to evaluate effectiveness of arenaviral vaccines. In these applications, a subject who has been immunized with a test vaccine against an arenavirus (e.g., LASV) is examined for production of antibodies against the virus, esp. neutralizing antibodies. For example, a blood sample can be taken from the subject, which can then be contacted with an recombinant arenavirus glycoproteins or glycoprotein trimer. Arenaviral specific antibodies that react with the immunogen (e.g. recombinant arenavirus glycoproteins, glycoprotein trimers) can then be assessed qualitatively and quantitatively. For example, the antigen-antibody immune complexes can be readily isolated from the blood sample. The types of the antibodies present in the blood sample can be analyzed qualitatively by comparing them to antibodies known in the art, e.g., all known LASV neutralizing antibodies. These can be accomplished by employing the various techniques that have been routinely practiced in the art or exemplified herein. Methods for analyzing and identifying antibodies are described in greater detail in Robinson et al., Nat. Comm. 7:11544, 2016; Hastie et al., J. Virol. 90:4556-62, 2016; Hastie et al., Science 356:923-928, 2017; Li et al., Vaccine. 355172-5178, 2017; Sommerstein et al., PLoS Pathog. 11:e1005276, 2015; Bukbuk et al., Trans. R. Soc. Trop. Med. Hyg. 108:768-73, 2014; and Shaffer et al., PLoS Negl. Trop. Dis. 8:e2748, 2014.; which are incorporated herein by reference in their entirety for all purposes. The recombinant arenavirus glycoproteins and glycoprotein trimers provided herein including embodiments thereof are contemplated to be useful for methods of quantitatively examining neutralizing antibodies produced in the subject as a result of vaccination. Such an analysis can also be readily performed with standard immunological protocols well known in the art (e.g., ELISA). By detection of virtually all neutralizing antibodies and also non-neutralizing antibodies, the engineered arenaviral immunogens of the invention thus enable both qualitative and quantitative evaluation of antibody responses elicited by a vaccine in a subject or a group of subjects.

The recombinant arenavirus glycoproteins and glycoprotein trimers provided herein including embodiments thereof can be used in evaluating the effectiveness of an arenavirus vaccine in a subject. In embodiments, the method includes contacting a biological sample from a subject who has been administered with a vaccine for an arenavirus with the recombinant arenavirus glycoproteins and glycoprotein trimers provided herein, (b) detecting antibodies in the biological sample that specifically bind to the recombinant arenavirus glycoproteins and glycoprotein trimers provided, and (c) performing quantitative and qualitative analysis of the antibodies detected in the biological sample, thereby evaluating effectiveness of the arenavirus vaccine in the subject.

The recombinant arenavirus glycoproteins and glycoprotein trimers provided herein including embodiments thereof can be provided as components of diagnostic kits. In embodiments, the kit includes components including packaging and reagents. In embodiments, the reagents include buffers, substrates, antibodies or ligands (e.g. control antibodies or ligands), and detection reagents. In embodiments, the kit includes an instruction sheet.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### P EMBODIMENTS

P embodiment 1: A recombinant arenavirus glycoprotein comprising an exogenous domain of an arenavirus glycoprotein polypeptide and an exogenous trimerization domain.

P embodiment 2: The recombinant arenavirus glycoprotein of embodiment 1, wherein the exogenous trimerization domain is 1NOG.

P embodiment 3: A recombinant arenavirus glycoprotein comprising three recombinant arenavirus glycoproteins non-covalently bound together, wherein each of the three recombinant arenavirus glycoproteins comprise an exogenous domain of an arenavirus glycoprotein and an exogenous trimerization domain.

P embodiment 4: A recombinant arenavirus glycoprotein comprising an exogenous domain of an arenavirus glycoprotein polypeptide and a C-terminal LPXTG motif.

P embodiment 5: The recombinant arenavirus glycoprotein of any of embodiments 1 to 4, wherein the exogenous domain of an arenavirus glycoprotein polypeptide comprises the polypeptide sequence of SEQ ID NO.:1.

P embodiment 6: The recombinant arenavirus glycoprotein of embodiment 1, wherein the exogenous domain of an arenavirus glycoprotein polypeptide comprises the mutations CysR4, G243C, 1350C, E329P and L258R/L259R.

P embodiment 7: The recombinant arenavirus glycoprotein of any of embodiments 1 to 6, wherein the exogenous domain of an arenavirus glycoprotein comprises a) mutations R193F, D211L, K339L, and H354L; or, b) mutations in a) and H3051, H305L, H305V or L128P.

P embodiment 8: The recombinant arenavirus glycoprotein of any of embodiments 1 to 7 comprising a signal peptide.

P embodiment 9: The recombinant arenavirus glycoprotein of any of embodiments 1 to 7 comprising a protease cleavage site.

P embodiment 10: The recombinant arenavirus glycoprotein of any of embodiments 1 to 7 comprising a protein purification tag.

P embodiment 11: The recombinant arenavirus glycoprotein of any of embodiments 1 to 7 comprising a linker region.

P embodiment 12: An antibody directed to any of the compositions of embodiments 1 to 11.

P embodiment 13: A vaccine comprising any of the compositions of c embodiments 1 to 11 and a pharmaceutically acceptable excipient.

P embodiment 14: The vaccine of embodiment 13, further comprising an adjuvant.

P embodiment 15: The vaccine of embodiment 14, wherein said adjuvant is a gel-type, microbial, particulate, oil-emulsion, surfactant-based, or synthetic adjuvant.

P embodiment 16: The vaccine of embodiment 13, wherein the adjuvant is aluminum hydroxide/phosphate, calcium phosphate, muramyl dipeptide (MDP), a bacterial exotoxin, an endotoxin-based adjuvant, a biodegradable adjuvant, polymer microspheres, immunostimulatory complexes (ISCOMs), liposomes, Freund's incomplete adjuvant, microfluidized emulsions, saponins, muramyl peptide derivatives, nonionic block copolymers, polyphosphazene (PCPP), synthetic polynucleotide, or a thalidomide derivative.

P embodiment 17: A nucleic acid encoding any of the compositions of embodiments 1 to 11.

P embodiment 18: The nucleic acid of embodiment 17, comprising an expression vector.

P embodiment 19: A method of treating an arenavirus infection to a subject in need, said method comprising administering a recombinant arenavirus glycoprotein of embodiments 1 to 11, an antibody of embodiment 12, or a vaccine of embodiments 13 to 16.

P embodiment 20: The method of embodiment 19, wherein the causative agent of the arenavirus infection is Lymphocytic choriomeningitis virus (LCMV), Junin virus, Machupo virus, Lassa virus, Guanarito virus, Sabia virus, Chapare virus, Whitewater Arroyo virus, or Lujo virus.

P embodiment 21: A method of preventing infection in a subject, said method comprising administering a vaccine of any of embodiments 13 to 16.

P embodiment 22: A method of generating arenavirus-specific antibodies, said method comprising administering any of the compositions of embodiments 1 to 11 to a subject, obtaining biological material from said subject, and purifying antibodies from said biological material.

P embodiment 23: A method for detecting arenavirus infection comprising contacting a biological sample with the antibody of embodiment 12 and detecting the presence or absence of arenavirus.

P embodiment 24: A method of determining the presence of antibodies specific for an arenavirus in a biological sample comprising contacting said biological sample with a composition of any of the embodiments 1 to 11 and detecting the presence or absence of arenavirus-specific antibody.

### EMBODIMENTS

Embodiment 1: A recombinant arenavirus glycoprotein comprising an arenavirus glycoprotein ectodomain and a trimerization domain.

Embodiment 2: The recombinant arenavirus glycoprotein of embodiment 1, wherein said ectodomain comprises an arenavirus GP1/GP2 protein, wherein said arenavirus GP1/GP2 protein comprises a GP1 domain and a GP2 domain.

Embodiment 3: The recombinant arenavirus glycoprotein of embodiment 2, wherein the C-terminus of the GP1 domain is bound to the N-terminus of the GP2 domain.

Embodiment 4: The recombinant arenavirus glycoprotein of embodiment 3, wherein said ectodomain further comprises a protease cleavage site, wherein said protease cleavage site is located between said GP1 domain and said GP2 domain.

Embodiment 5: The recombinant arenavirus glycoprotein of any one of embodiments 2-4, wherein said arenavirus GP1/GP2 protein is a stabilized arenavirus GP1/GP2 protein.

Embodiment 6: The recombinant arenavirus glycoprotein of embodiment 5, wherein said stabilized arenavirus GP1/GP2 protein comprises a disulfide bond between a first cysteine amino acid side chain in the GP1 domain and a second cysteine amino acid side chain in the GP2 domain.

Embodiment 7: The recombinant arenavirus glycoprotein of embodiment 6, wherein said first cysteine amino acid side chain in the GP1 domain is at a position corresponding to position 207 of SEQ ID NO:18 and said second cysteine amino acid side chain in the GP2 domain is at a position corresponding to position 360 of SEQ ID NO:18.

Embodiment 8: The recombinant arenavirus glycoprotein of any one of embodiments 2-7, wherein said trimerization domain is bound to the C-terminus of said GP2 domain.

Embodiment 9: The recombinant arenavirus glycoprotein of embodiment 8, wherein said trimerization domain is covalently bound to said C-terminus of said GP2 domain though a peptide linker.

Embodiment 10: The recombinant arenavirus glycoprotein of embodiment 9, wherein said peptide linker is from about 5 to about 80 amino acid residues in length.

Embodiment 11: The recombinant arenavirus glycoprotein of embodiment 9, wherein said peptide linker is from about 8 to about 30 amino acid residues in length.

Embodiment 12: The recombinant arenavirus glycoprotein of any one of embodiments 1-10, wherein said trimerization domain is a protein having a molecular weight of about 2 kDa to about 50 kDa.

Embodiment 13: The recombinant arenavirus glycoprotein of embodiment 12, wherein said trimerization domain is a protein having a molecular weight of about 3 kDa to about 30 kDa.

Embodiment 14: The recombinant arenavirus glycoprotein of any one of embodiments 1-13, wherein said trimerization domain is a leucine zipper, 1NOG, Fibritin, or GCN4.

Embodiment 15: The recombinant arenavirus glycoprotein of embodiment 14, wherein said trimerization domain is 1NOG.

Embodiment 16: The recombinant arenavirus glycoprotein of any one of embodiments 1-15, wherein said ectodomain comprises: an arginine at a position corresponding to position 258 of SEQ ID NO:18, an arginine at a position corresponding to position 259 of SEQ ID NO:18, and a proline at a position corresponding to position 329 of SEQ ID NO:18.

Embodiment 17: The recombinant arenavirus glycoprotein of any of embodiments 1-16, wherein said ectodomain comprises: a phenylalanine at a position corresponding to position 193 of SEQ ID NO:18, a leucine at a position corresponding to position 211 of SEQ ID NO:18, a leucine at a position corresponding to position 339 of SEQ ID NO:18, and a leucine at a position corresponding to position 354 of SEQ ID NO:18.

Embodiment 18: The recombinant arenavirus glycoprotein of any of embodiments 1-17, wherein said ectodomain further comprises: a proline at a position corresponding to position 128 of SEQ ID NO:18, or an isoleucine, leucine or valine at a position corresponding to position 305 of SEQ ID NO:18.

Embodiment 19: The recombinant arenavirus glycoprotein of any of embodiments 1-16, wherein said ectodomain comprises an amino acid sequence having a sequence identify of at least 80% to the amino acid sequence of SEQ ID NO:27.

Embodiment 20: The recombinant arenavirus glycoprotein of any of embodiments 2-9, wherein said ectodomain further comprises a signal peptide.

Embodiment 21: The recombinant arenavirus glycoprotein of embodiment 20, wherein said signal peptide is covalently bound to the N-terminus of the GP1 domain.

Embodiment 22: The recombinant arenavirus glycoprotein of embodiment 20 or 21, wherein said signal peptide comprises the sequence of SEQ ID NO:2.

Embodiment 23: The recombinant arenavirus glycoprotein of any of embodiments 1-22, wherein said ectodomain is non-glycosylated.

Embodiment 24: The recombinant arenavirus glycoprotein of any of embodiments 1-23, wherein said ectodomain is glycosylated.

Embodiment 25: The recombinant arenavirus glycoprotein of embodiment 24, wherein the ectodomain is glycosylated at one or more amino acid residues at positions corresponding to 79, 89, 99, 119, 365 and 373 of SEQ ID NO:18.

Embodiment 26: The recombinant arenavirus glycoprotein of any of embodiments 1-23, wherein said ectodomain is not glycosylated at one or more amino acid residues at positions corresponding to 390 or 395 of SEQ ID NO:18.

Embodiment 27: A glycoprotein trimer comprising three of the recombinant arenavirus glycoproteins of any one of embodiments 1-26, wherein said three recombinant arenavirus glycoproteins are bound by non-covalent attachment of the trimerization domains.

Embodiment 28: A nucleic acid encoding the recombinant arenavirus glycoprotein of any of embodiments 1-26.

Embodiment 29: The nucleic acid of embodiment 28, further comprising a vector.

Embodiment 30: A cell comprising the recombinant arenavirus glycoprotein of any one of claims 1-26 or the glycoprotein trimer of embodiment 27.

Embodiment 31: A cell comprising the nucleic acid of embodiment 28 or 29.

Embodiment 32: The cell of embodiment 30 or 31, wherein the cell is a human cell.

Embodiment 33: A vaccine composition comprising the recombinant arenavirus glycoprotein of any one of embodiments 1-26 and a pharmaceutically acceptable excipient.

Embodiment 34: A vaccine composition comprising the glycoprotein trimer of embodiment 27 and a pharmaceutically acceptable excipient.

Embodiment 35: The vaccine composition of embodiment 33 or 34, further comprising an adjuvant.

Embodiment 36: A method of treating or preventing a viral disease in a subject in need of such treatment or prevention, said method comprising administering a therapeutically or prophylactically effective amount of the recombinant arenavirus glycoprotein of any one of claims 1-26 to said subject.

Embodiment 37: A method of treating or preventing a viral disease in a subject in need of such treatment or prevention, said method comprising administering a therapeutically or prophylactically effective amount of the glycoprotein trimer of embodiment 27 to said subject.

Embodiment 38: The method of embodiment 36 or 27, wherein said viral disease is caused by Lymphocytic choriomeningitis virus (LCMV), Junin virus, Machupo virus, Lassa virus, Guanarito virus, Sabia virus, Chapare virus, Whitewater Arroyo virus, or Lujo virus.

Embodiment 39: A method for immunizing a subject susceptible to a viral disease, comprising administering the recombinant arenavirus glycoprotein of any one of embodiments 1-26 to a subject under conditions such that antibodies directed to said arenavirus glycoprotein or a fragment thereof are produced.

Embodiment 40: A method for immunizing a subject susceptible to a viral disease, comprising administering the glycoprotein trimer of embodiment 27 to a subject under conditions such that antibodies directed to said glycoprotein trimer or a fragment thereof are produced.

Embodiment 41: A method of diagnosing arenavirus infection in a subject, the method comprising: (a) contacting a biological sample obtained from the subject with the recombinant arenavirus glycoprotein of any one of claims 1-26, and (b) detecting binding of one or more antibodies to said recombinant arenavirus glycoprotein, thereby diagnosing arenavirus infection in said subject.

Embodiment 42: A method of diagnosing arenavirus infection in a subject, the method comprising: (a) contacting a biological sample obtained from the subject with the glycoprotein trimer of claim 27, and (b) detecting binding of one or more antibodies to said glycoprotein trimer, thereby diagnosing arenavirus infection in said subject.

Embodiment 43: A method for evaluating effectiveness of an arenavirus vaccine in a subject, the method comprising (a) contacting a biological sample from a subject who has been administered with the vaccine composition of any one of embodiments 33-35, (b) detecting antibodies in the biological sample that bind to the recombinant arenavirus glycoprotein or glycoprotein trimer, and (c) performing quantitative and qualitative analysis of the antibodies detected in the biological sample, thereby evaluating effectiveness of the arenavirus vaccine in the subject.

### EXAMPLES

Lassa virus glycoprotein (GP) is typically synthesized as a precursor including a signal peptide, GP1, and GP2. The precursor is usually processed to cleave the signal peptide from GP1 and GP2. In instances, the GP is further processed into an N-terminal subunit (GP1) and a C-terminal subunit (GP2) by way of a protease which targets a cleavage site between GP1 and GP2 of the virion protein. Typically, processing (e.g. cleavage) of the GP allows for formation of a non-covalently associated trimer of dimers, each dimer including GP1 and GP2. The GP trimer (e.g. the complex including GP1 and GP2 dimers) is typically required for mediating viral entry to host cells. The virion GP complex includes GP1, which is thought to be responsible for receptor binding, and GP2, which typically includes a transmembrane portion and a fusion-mediating portion. In some instances, the mature virion glycoprotein trimer may further include a signal peptide (SSP). Usually, the virion trimeric complex fuses with the host cell membrane, thereby enabling entry into the host cell.

Applicant has generated a genetically modified construct which allowed production of high quantities of a fully processed pre-fusion GP trimer that is recognized by neutralizing antibodies but not by antibodies specific for the post-fusion form of GP, which adopts a different structural configuration. Applicant first generated a modified GP, termed GPCysR4, which is monomeric in solution. The monomeric modified GP includes the GP ectodomain (e.g. surface portions of GP1 and GP2), and a protease cleavage site between the GP1 and GP2 domains. Applicant generated mutations in GP1 and GP2, which allowed disulfide bond interactions between the GP1 and GP2 subunits following cleavage of the protease site located between the GP1 and GP2 subunits. The mutations thereby increased formation of a GP1/GP2 dimer. The disulfide bonds additionally stabilized the GP1/GP2 complex, by allowing formation of covalent disulfide bonds.

Applicants then attached a trimerization domain (e.g. 1NOG) to GPCysR4. This allowed for formation of a trimer, each monomer of the trimer including GP1/GP2 and the trimerization domain.

### Example 1: Antibody Discovery

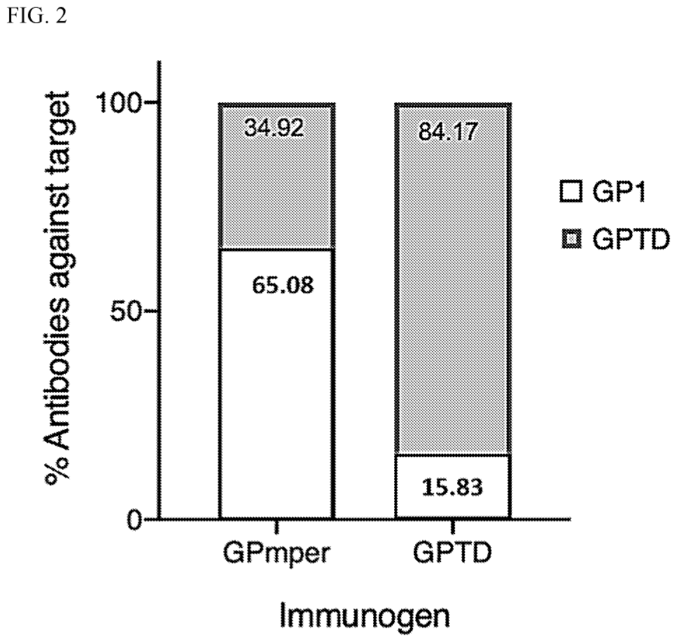

Mice were immunized with an unmodified LASV GP (called GPmper) or the prefusion-trimeric GP (GPTD) (SEQ ID NO:1), which includes three monomers comprising GP1/GP2 and the 1NOG trimerization domain. Using the novel Beacon platform, which allows for real-time single-cell interrogation of plasma cells and identification of antigen-specificity, it was shown that immunization with the trimeric form of Lassa GP produces a greater number of trimer-specific antibodies, which are highly neutralizing but hard to elicit, and fewer antibodies targeting the GP1 subunit alone. Antibodies that target the GP1 subunit are known to contain very few, perhaps only a single, neutralizing epitopes. Thus, results illustrated in FIG. 2 show that the antibody response can be directed by the chosen immunogen.

### Example 2: GPCysR4-TD Trimer

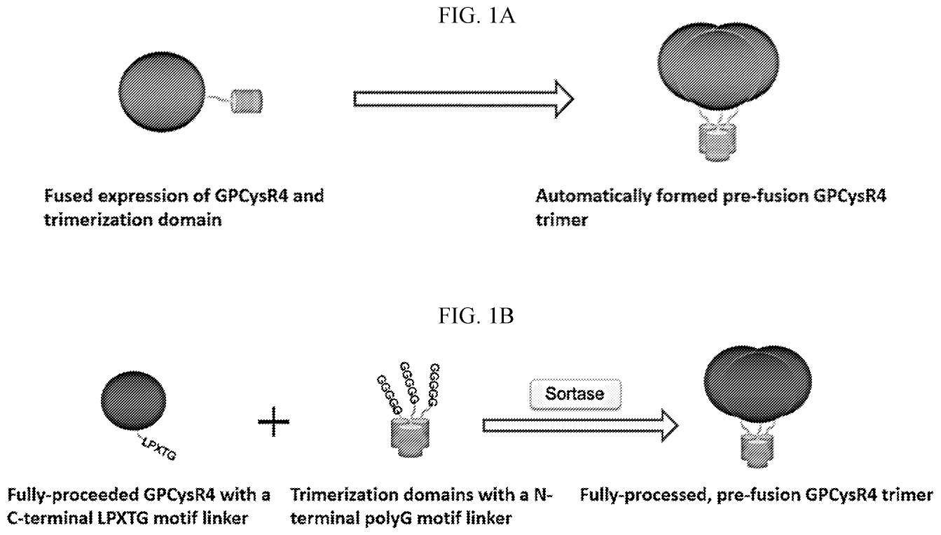

Fused expression of the ectodomain of GPCysR4 (SEQ ID NO:3) with a heterologous C-terminal trimerization domain (e.g. 1NOG, a trimerization domain identified from Thermoplasma acidophilum) (3) (SEQ ID NO:5) gave rise to GP trimer formation (FIG. 1A).

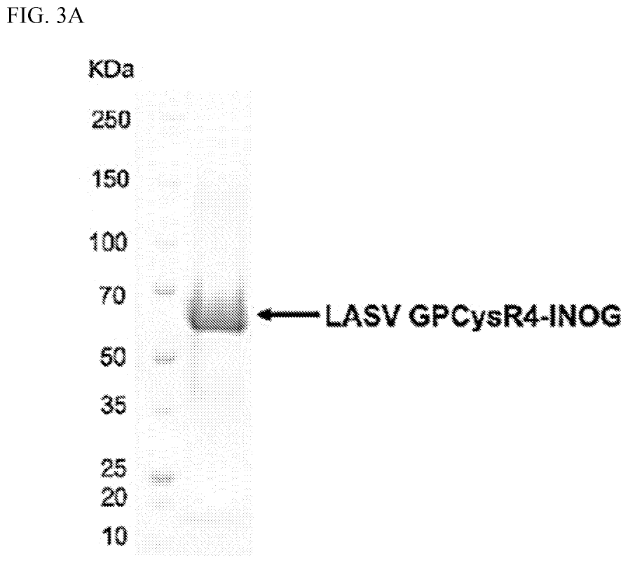

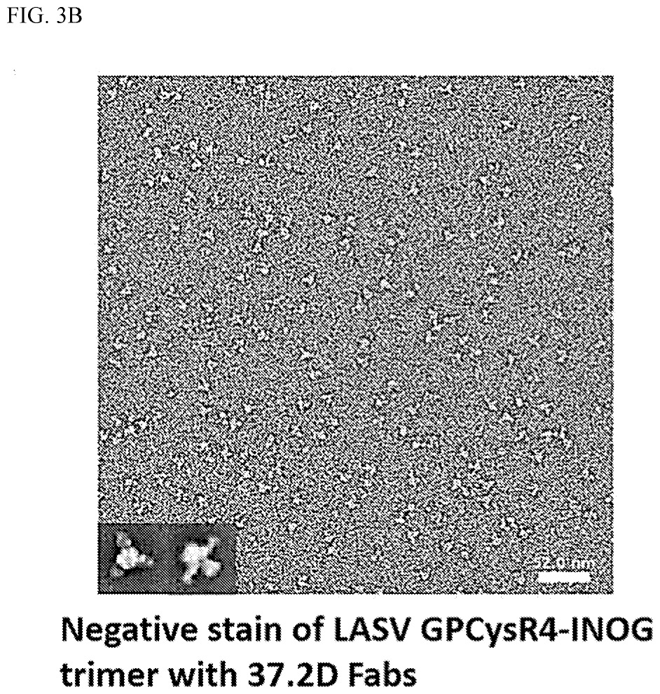

The GPCysR4 of LASV fused with the 1NOG trimerization domain was expressed, generating the protein of SEQ ID NO:1, and subsequently purified from Drosophila S2 expression system (FIG. 3A). The LASV GPCysR4-1NOG formed GP trimer was able to be recognized by Fab fragments of 37.2D, a neutralizing antibody that only recognizes the pre-fusion GP trimer (FIG. 3B).

### Example 3: Sortase-Mediated Fully-Processed GP Trimer Formation

Highly stable and homogeneous GP trimers that were ˜90% processed (termed GPCysR4-TD) were previously developed. This protein is suitable for new antibody discovery, but lacks the homogeneity required for structural biology work. Hence, engineering efforts were focused on the production of stabilized GP trimer that is 100% processed, which was achieved by the sortase-modified strategy described herein.

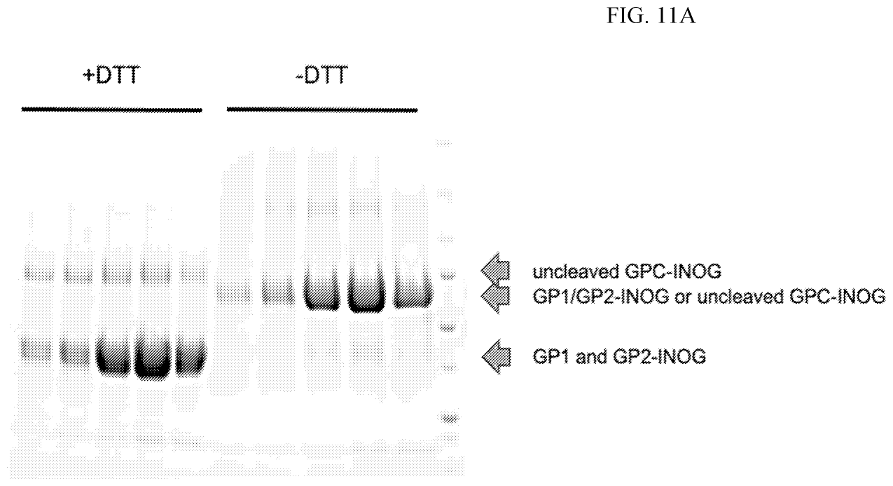

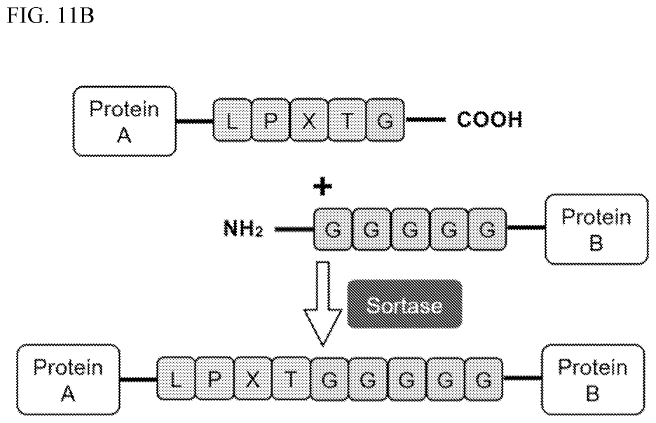

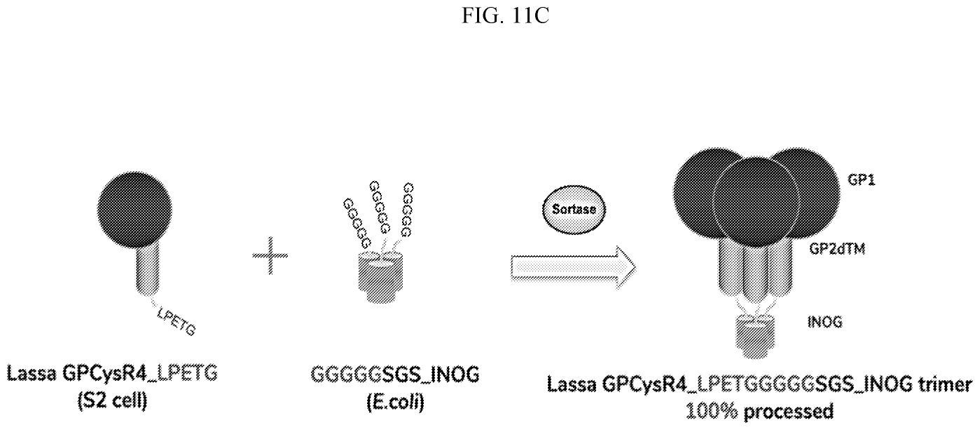

Sortases are a group of prokaryotic enzymes which catalyze transpeptidation reactions. Staphylococcus aureus sortase recognizes the motif LPXTG (Leu-Pro-any-Thr-Gly) (SEQ ID NO:15), cleaves the peptide bond between T and G, and catalyzes a new peptide bond formation between the C-terminal T and a G at the N-terminus of another protein (4) (FIG. 11B). The LPXTG motif was introduced to the C-terminus of the ectodomain of GPCysR4 (SEQ ID NO:3), and a poly G sequence was labeled to the N-terminus of the trimerization domain, for example 1NOG (SEQ ID NO:11). The engineered GPCysR4-LPXTG monomer (SEQ ID NO:10) and the heterologous trimerization domain were expressed and purified separately, and covalently joined by the sortase-mediated transpeptidation to form GPCysR4-LPXTG-TD trimers (FIG. 1B).

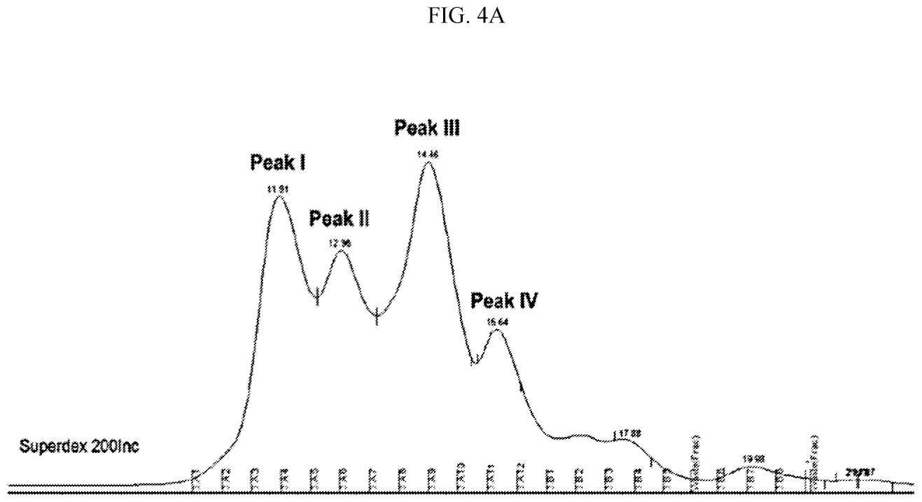

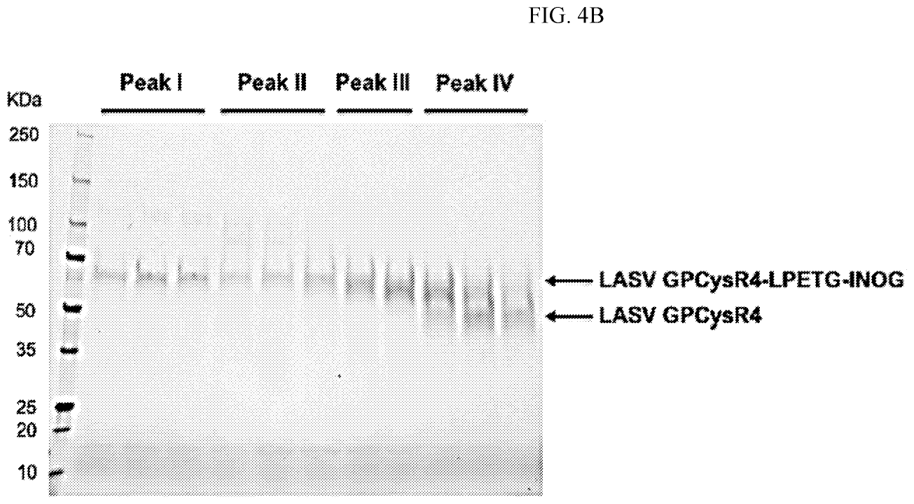

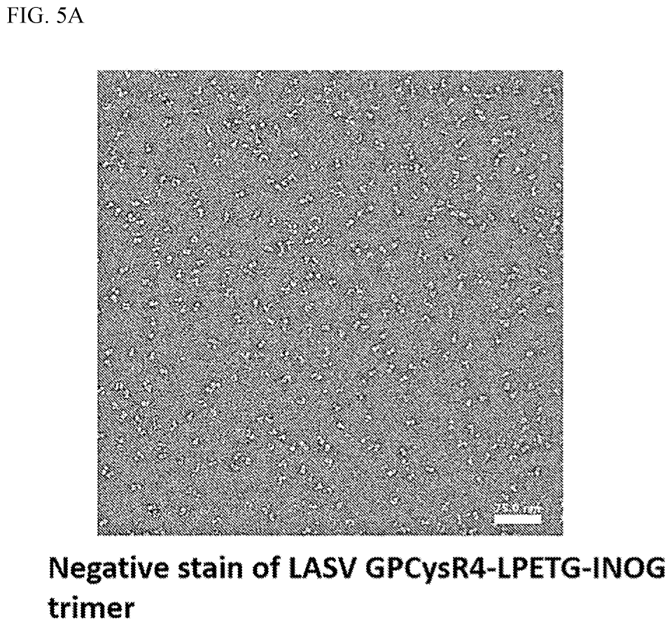

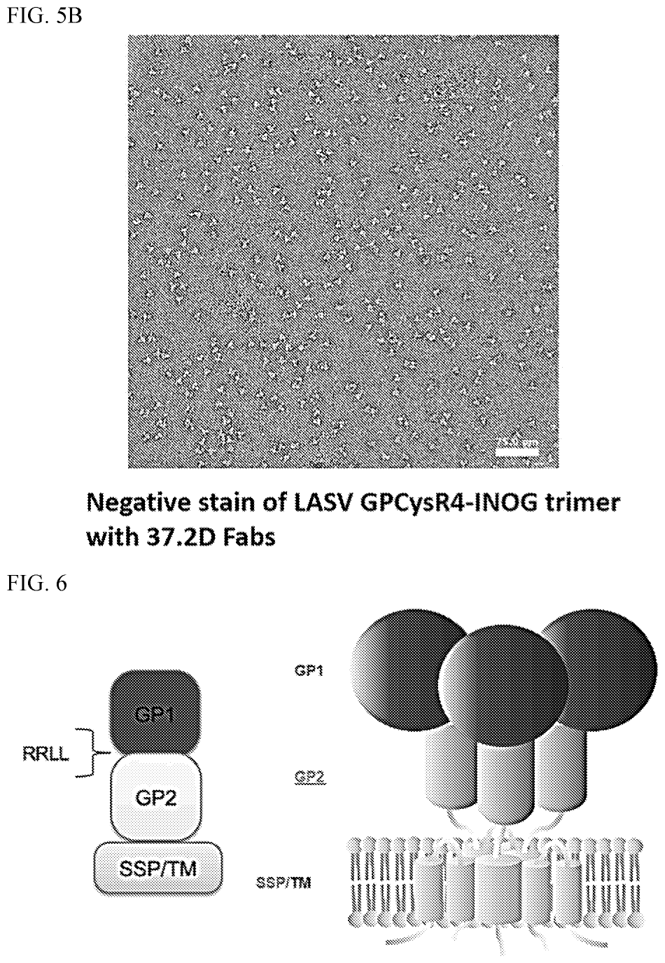

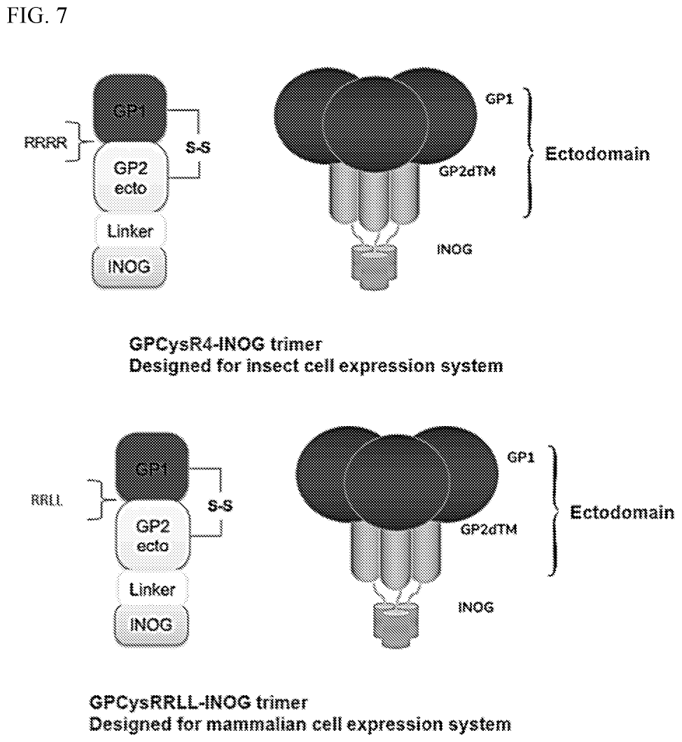

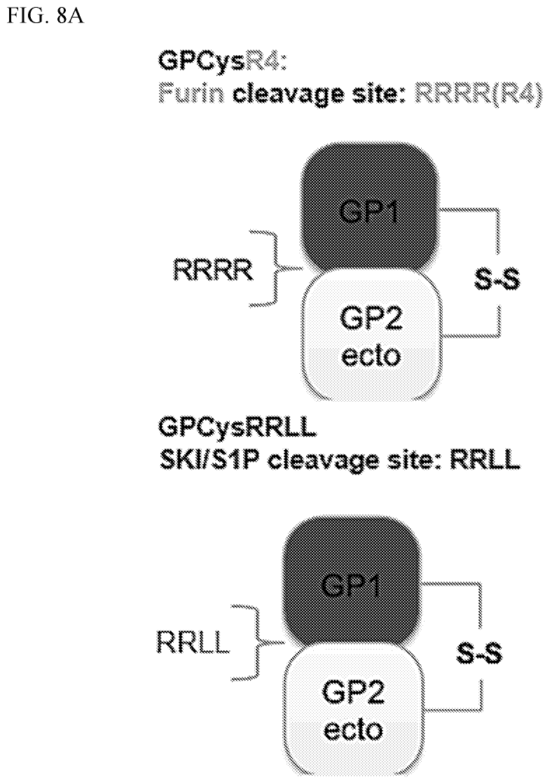

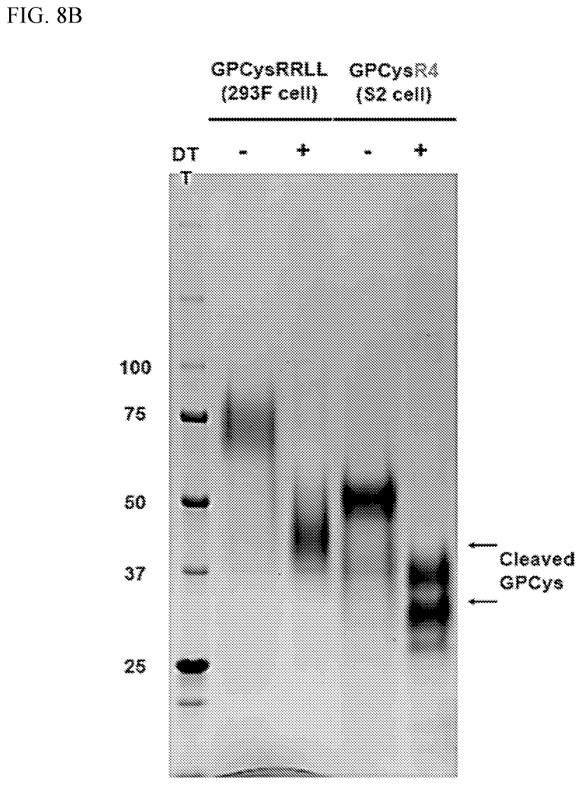

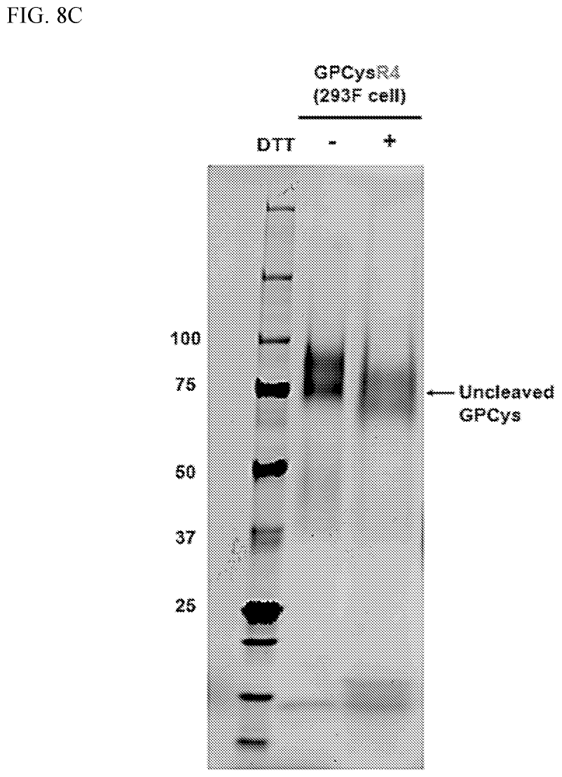

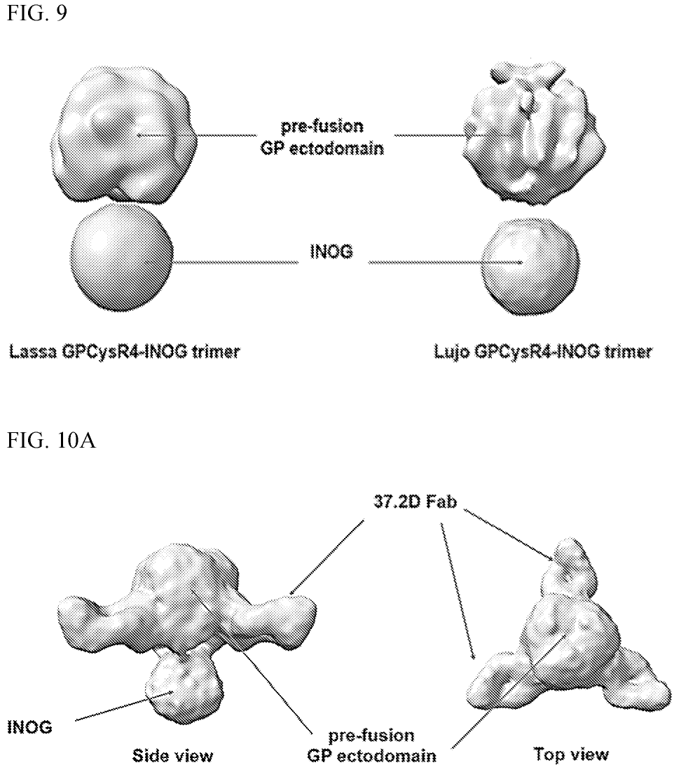

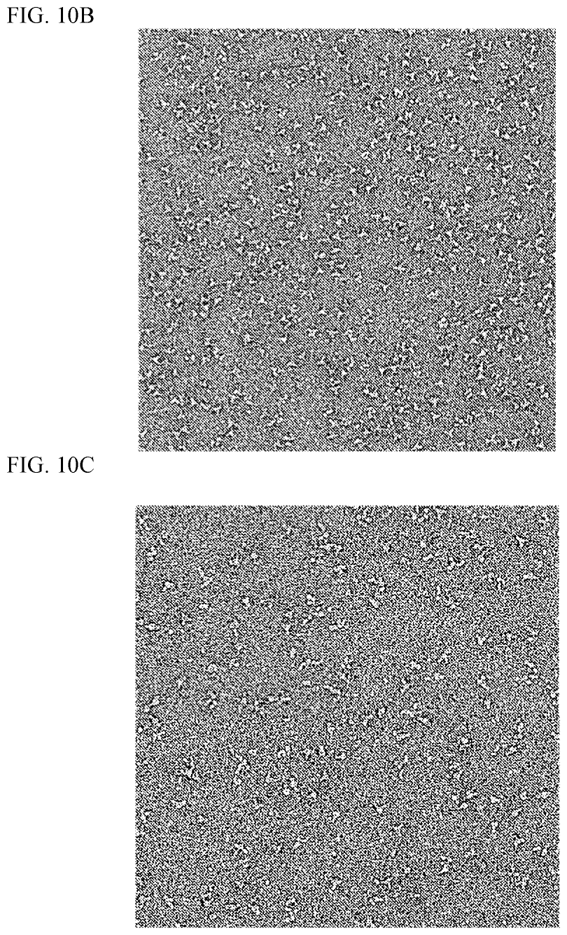

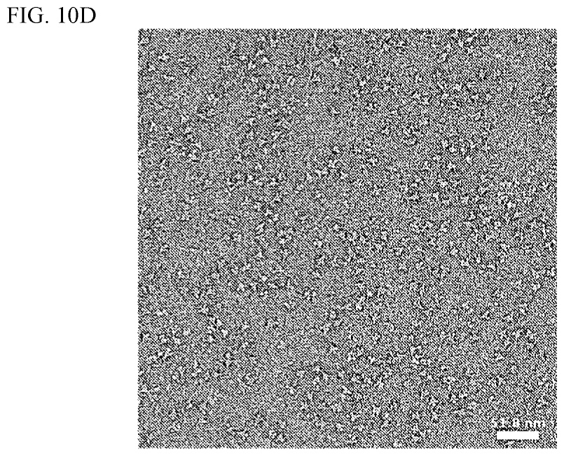

The LASV GPCysR4 with C terminus LPETG (SEQ ID NO:14) motif (LASV GPCysR4-LPETG) and the 1NOG with N terminus poly G motif (GGGGG-1NOG) (SEQ ID NO:11) were purified from S2 cells and E. coli respectively. LASV GPCysR4-LPETG-1NOG trimers were formed by catalysis of the sortase A and were further purified by the S200Inc size exclusion column (FIG. 4A). Negative stain EM demonstrates that LASV GPCysR4-LPETG-1NOG, from Peak I in FIG. 4A, is indeed trimeric (FIG. 5A), and can be recognized by 37.2D Fabs (FIG. 5B).

### Example 4: Optimization and Antigenicity of Pre-Fusion Trimer Proteins

Immunization studies determine the antigenicity of prefusion stabilized LASV GP trimers as compared to wild-type, unmodified LASV GP.

Further, the GPs and trimerization domains of LASV and other arenaviruses are engineered and optimized for activity and stability. The linker connecting the GP and timerization domains are additionally optimized.

These studies achieve constructs for antibody and vaccine discovery, in addition to structural studies for additional antibody and therapeutic development.

### REFERENCES

1. J. E. Robinson et al., Most neutralizing human monoclonal antibodies target novel epitopes requiring both Lassavirus glycoprotein subunits. Nat Commun 7, 11544 (2016).
2. K. M. Hastie et al., Structural basis for antibody-mediated neutralization of Lassa virus. Science 356, 923-928(2017).
3. V. Saridakis et al., The structural basis for methylmalonic aciduria. The crystal structure of archaeal ATP:cobalamin adenosyltransferase. J Biol Chem 279, 23646-23653 (2004).
4. I. Chen, B. M. Dorr, D. R. Liu, A general strategy for the evolution of bond-forming enzymes using yeast display. Proc Natl Acad Sci USA 108, 11399-11404 (2011).

INFORMAL SEQUENCE LISTING
(GPTD; trimer-stabilized GP; Includes Signal Peptide (SEQ ID NO: 2),
point mutations R207C, G360C, E329P and L258R/L259R, ectodomain that stabilizes the GP in
a trimeric state (SEQ ID NO: 5), Enterokinase cleavage site (SEQ ID NO: 6) to remove the double
strepII tag used for purification (SEQ ID NO: 7))
SEQ ID NO: 1
MGQIVTFFQEVPHVIEEVMNIVLIALSVLAVLKGLYNFATCGLVGLVTFLLLCGRS
CTTSLYKGVYELQTLELNMETLNMTMPLSCTKNNSHHYIMVGNETGLELTLTNTSIINH
KFCNLSDAHKKNLYDHALMSIISTFHLSIPNFNQYEAMSCDFNGGKISVQYNLSHSYAG
DAANHCGTVANGVLQTFMRMAWGGSYIALDSGCGNWDCIMTSYQYLIIQNTTWEDHC
QFSRPSPIGYLGLLSQRTRDIYISRRRRGTFTWTLSDSEGKDTPGGYCLTRWMLIEAELK
CFGNTAVAKCNEKHDEEFCDMLRLFDFNKQAIQRLKAPAQTSIQLINKAVNALINDQLI
MKNHLRDIMCIPYCNYSKYWYLNHTTTGRTSLPKCWLVSNGSYLNETHFSDDIEQQAD
NMITEMLQKEY MERQGKTPLGLVDLEGGSPVVEVQGTIDELNSFIGYALVLSRWDDIRN
DLFRIQNDLFVLGEDVSTGGKGRTVTREMIDYLEARVKEMKAEIGKIELFVVPGGSVESASLH
MARAVSRRLERRIVAASKLTEINKNVLIYANRLSSILFMHALISNKRLNIPEKIWA LEVDDDDK
AGWSHPQFEKGGGSGGGSGGGSWSHPQFEK*
(Signal Peptide)
SEQ ID NO: 2
MGQIVTFFQEVPHVIEEVMNIVLIALSVLAVLKGLYNFATCGLVGLVTFLLLCGRS
CT
(cysteine-stabilized GP (GPCysR4); sequence comprises the point
mutations R207C, G360C, E329P and L258R/L259R (relative to the sequence of SEQ ID
NO: 13))
SEQ ID NO: 3
TSLYKGVYELQTLELNMETLNMTMPLSCTKNNSHHYIMVGNETGLELTLTNTSIINHKF
CNLSDAHKKNLYDHALMSIISTFHLSIPNFNQYEAMSCDFNGGKISVQYNLSHSYAGDA
ANHCGTVANGVLQTFMRMAWGGSYIALDSGCGNWDCIMTSYQYLIIQNTTWEDHCQF
SRPSPIGYLGLLSQRTRDIYISRRRRGTFTWTLSDSEGKDTPGGYCLTRWMLIEAELKCFG
NTAVAKCNEKHDEEFCDMLRLFDFNKQAIQRLKAPAQTSIQLINKAVNALINDQLIMKN
HLRDIMCIPYCNYSKYWYLNHTTTGRTSLPKCWLVSNGSYLNETHFSDDIEQQADNMIT
EMLQKEY MERQGKTPLGLVD
(non-specific linker sequence)
SEQ ID NO: 4
MERQGKTPLGLVD
(Ectodomain that stabilizes the GP in a trimeric state, ATP:cobalamin
adenosyltransferase MMAB from Thermoplasma acidophilum (PDB code INOG))
SEQ ID NO: 5
PVVEVQGTIDELNSFIGYALVLSRWDDIRNDLFRIQNDLFVLGEDVSTGGKGRTVTREMI
DYLEARVKEMKAEIGKIELFVVPGGSVESASLHMARAVSRRLERRIVAASKLTEINKNV
LIYANRLSSILFMHALISNKRLNIPEKIWA
(Enterokinase cleavage site which may be used to remove the double
strepII tag used for purification)
SEQ ID NO: 6
DDDDK
(double strepII tag used for purification)
SEQ ID NO: 7
WSHPQFEK
(AVI-tag for site-specific biotinylation)
SEQ ID NO: 8
GLNDIFEAQKIEWHE
(HRV3C protease cleavage site)
SEQ ID NO: 9
LEVLFQGP
(GPCysR4-LPXTG)
SEQ ID NO: 10
MGQIVTFFQEVPHVIEEVMNIVLIALSVLAVLKGLYNFATCGLVGLVTFLLLCGRS
CTTSLYKGVYELQTLELNMETLNMTMPLSCTKNNSHHYIMVGNETGLELTLTNTSIINH
KFCNLSDAHKKNLYDHALMSIISTFHLSIPNFNQYEAMSCDFNGGKISVQYNLSHSYAG
DAANHCGTVANGVLQTFMRMAWGGSYIALDSGCGNWDCIMTSYQYLIIQNTTWEDHC
QFSRPSPIGYLGLLSQRTRDIYISRRRRGTFTWTLSDSEGKDTPGGYCLTRWMLIEAELKC
FGNTAVAKCNEKHDEEFCDMLRLFDFNKQAIQRLKAPAQTSIQLINKAVNALINDQLIM
KNHLRDIMCIPYCNYSKYWYLNHTTTGRTSLPKCWLVSNGSYLNETHFSDDIEQQADN
MITEMLQKEYLPETG LVDLEVDDDDKAGWSHPQFEKGGGSGGGSGGGSWSHPQFEK*
(Trimerization domain (1NOG))
SEQ ID NO: 11
GGGGGSGSPVVEVQGTIDELNSFIGYALVLSRWDDIRNDLFRIQNDLFVLGEDVSTGGK
GRTVTREMIDYLEARVKEMKAEIGKIELFVVPGGSVESASLHMARAVSRRLERRIVAAS
KLTEINKNVLIYANRLSSILFMHALISNKRLNIPEKIWA LEVHHHHHHGSGGLNDIFEAQ
KIEWHE
(linker)
SEQ ID NO: 12
LVDLEV
(Lassa virus pre-glycoprotein GP- Uniprot P08669)
SEQ ID NO: 13
MGQIVTFFQEVPHVIEEVMNIVLIALSVLAVLKGLYNFATCGLVGLVTFLLLCGRS
CTTSLYKGVYELQTLELNMETLNMTMPLSCTKNNSHHYIMVGNETGLELTLTNTS
IINHKFCNLSDAHKKNLYDHALMSIISTFHLSIPNFNQYEAMSCDFNGGKISVQYNLS
HSYAGDAANHCGTVANGVLQTFMRMAWGGSYIALDSGRGNWDCIMTSYQYLIIQ
NTTWEDHCQFSRPSPIGYLGLLSQRTRDIYISRRLLGTFTWTLSDSEGKDTPGGYCLT
RWMLIEAELKCFGNTAVAKCNEKHDEEFCDMLRLFDFNKQAIQRLKAEAQMSIQLINK
AVNALINDQLIMKNHLRDIMGIPYCNYSKYWYLNHTTTGRTSLPKCWLVSNGSYLNET
HFSDDIEQQADNMITEMLQKEYMERQGKTPLGLVDLFVFSTSFYLISIFLHLVKIPTHRHI
VGKSCPKPHRLNHMGICSCGLYKQPGVPVKWKR
(peptide motif)
SEQ ID NO: 14
LPETG
(peptide motif)
SEQ ID NO: 15
LPXTG
(Lassa GPCysR4-INOG sequence of the C-ter of GP2, linker, and N-
ter of INOG)
SEQ ID NO: 16
MLQKEYMERQGKTPLGLVDLEGGSPVVEVQGT
(Lassa GPCysR4-LPETGGGGGSGS-INOG sequence of the C-ter of
GP2, linker, and N-ter of INOG)
SEQ ID NO: 17
MLQKEYLPETGGGGGSGSVVEVQGT
(signal peptide, GP1 and GP2)
SEQ ID NO: 18
MGQIVTFFQEVPHVIEEVMNIVLIALSVLAVLKGLYNFATCGLVGLVTFLLLCGRS
CTTSLYKGVYELQTLELNMETLNMTMPLSCTKNNSHHYIMVGNETGLELTLTNTSIINH
KFCNLSDAHKKNLYDHALMSIISTFHLSIPNFNQYEAMSCDFNGGKISVQYNLSHSYAG
DAANHCGTVANGVLQTFMRMAWGGSYIALDSGCGNWDCIMTSYQYLIIQNTTWEDHC
QFSRPSPIGYLGLLSQRTRDIYISRRRRGTFTWTLSDSEGKDTPGGYCLTRWMLIEAELK
CFGNTAVAKCNEKHDEEFCDMLRLFDFNKQAIQRLKAPAQTSIQLINKAVNALINDQLI
MKNHLRDIMCIPYCNYSKYWYLNHTTTGRTSLPKCWLVSNGSYLNETHFSDDIEQQAD
NMITEMLQKEY
(TEV cleavage site)
SEQ ID NO: 19
ENLYFQG
(TEV cleavage site)
SEQ ID NO: 20
ENLYFQS
(Thrombin cleavage site)
SEQ ID NO: 21
LVPRGS
(Furin cleavage site; X is a hydrophobic amino acid)
SEQ ID NO: 22
X-Arg-X-Lys-Arg-Arg-X
(S1P cleavage site)
SEQ ID NO: 23
RRSKLL
(S1P cleavage site; LCMV)
SEQ ID NO: 24
RRLA
(S1P cleavage site; Lujo virus)
SEQ ID NO: 25
RKLM
(S1P cleavage site; Junin virus)
SEQ ID NO: 26
RSLK
(GP1 and GP2 with stabilizing mutations)
SEQ ID NO: 27
TSLYKGVYELQTLELNMETLNMTMPLSCTKNNSHHYIMVGNETGLELTLTNTSIINHKF
CNLSDAHKKNLYDHALMSIISTFHLSIPNFNQYEAMSCDFNGGKISVQYNLSHSYAGDA
ANHCGTVANGVLQTFMRMAWGGSYIALDSGCGNWDCIMTSYQYLIIQNTTWEDHCQF
SRPSPIGYLGLLSQRTRDIYISRRRRGTFTWTLSDSEGKDTPGGYCLTRWMLIEAELKCFG
NTAVAKCNEKHDEEFCDMLRLFDFNKQAIQRLKAPAQTSIQLINKAVNALINDQLIMKN
HLRDIMCIPYCNYSKYWYLNHTTTGRTSLPKCWLVSNGSYLNETHFSDDIEQQADNMIT
EMLQKEY
(GP1 with stabilizing mutations)
SEQ ID NO: 28
TSLYKGVYELQTLELNMETLNMTMPLSCTKNNSHHYIMVGNETGLELTLTNTSIINHKF
CNLSDAHKKNLYDHALMSIISTFHLSIPNFNQYEAMSCDFNGGKISVQYNLSHSYAGDA
ANHCGTVANGVLQTFMRMAWGGSYIALDSGCGNWDCIMTSYQYLIIQNTTWEDHCQF
SRPSPIGYLGLLSQRTRDIYISRR
(GP2 with stabilizing mutations)
SEQ ID NO: 29
RRGTFTWTLSDSEGKDTPGGYCLTRWMLIEAELKCFGNTAVAKCNEKHDEEFCDMLRL
FDFNKQAIQRLKAPAQTSIQLINKAVNALINDQLIMKNHLRDIMCIPYCNYSKYWYLNH
TTTGRTSLPKCWLVSNGSYLNETHFSDDIEQQADNMITEMLQKEY
(Furin cleavage site)
SEQ ID NO: 30
RRRR
(SP1 cleavage site)
SEQ ID NO: 31
RRLL
(Linker including C-terminus of GP2 and N-terminus of INOG)
SEQ ID NO: 32
MLQKEYMERQGGGSPVVEVQGT
(Linker including C-terminus of GP2 and N-terminus of INOG)
SEQ ID NO: 33
MLQKEYMERQGKTPAPAPVVEVQGT
(Linker including C-terminus of GP2 and N-terminus of INOG)
SEQ ID NO: 34
MLQKEYMERQGKTPAPAPGGSPVVEVQGT
(Lassa GP sequence Lineage 1 (AAF86701.1))
SEQ ID NO: 35
mgqiitffqe vphvieevmn ivlialslla ilkglyniat cgiiglvafl flcgkscslt lkggyelqtl elnmetlnmt
mplsctknss hhyirvgnet gleltltnts iinhkfcnls dahkknlydh almsiistfh lsipnfnqye amscdfnggk
isvqynlshs yagdaaehcg tvangvlqtf mrmawggryi aldsgkgnwd cimtsyqyli iqnttwedhc qfsrpspigy
lgllsqrtrd iyisrrllgt ftwtlsdseg netpggyclt rwmlieaelk cfgntavakc nekhdeefcd mlrlfdfnkq
airrlkaeaq msiqlinkav nalindqlim knhlrdimgi rqgktplglv dififstsfy lisiflhlik ipthrhivgk
pcpkphrlnh mgvcscglyk hpgvptkwkr
(Lassa GP sequence Lineage 2 (AVO03605.1)
SEQ ID NO: 36
mgqiitffqe vphvieevmn ivlialslla ilkgiynvat cglfglvsfl llcgrscstt ykgvyelqtl eldmaslnmt
mplsctknns hhyimvgnet gleltltnts iinhkfcnls dahkknlydh almsiistfh lsipnfnqye amscdfnggk
isvqynlsht yavdaanhcg tiangvlqtf mrmawggsyi aldsgkgswd cimtsyqyli iqnttwedhc qfsrpspigy
lgllsqrtrd iyisrrllgt ftwtlsdseg netpggyclt rwmlieaelk cfgntavakc nekhdeefcd mlrlfdfnkq
airrlkteaq msiqlinkav nalindqlim knhlrdimgi pycnyskywy lnhtvtgrts lprcwlvsng sylnethfsd
dieqqadnmi tellqkeyid rqgktplglv dlfvfstsfy lisiflhlik ipthrhvigk pcpkphrlnh mgicscglyk
hpgvpvkwkr
(Lassa GP sequence Lineage 3 (AVO03595.1)
SEQ ID NO: 37
mgqiitffqe vphvieevmn ivlialslla ilkgiyniat cglfglisfl llcgrscstt ykgvyelqtl eldmanlnmt
mplsctknns hhyimvgnet gleltltnts iinhkfcnls dahkknlydh almsiistfh lsipnfnqye amscdfnggk
isvqynlshs yavdaaghcg tiangvlqtf mrmawggsyi aldsgkgnwd cimtsyqylv iqnttwedhc qfsrpspigy
lgllsqrtrd iyisrrllgt ftwtlsdseg neapggyclt rwmlieaelk cfgntaiakc nekhdeefcd mlrlfdfnkq
airrlkaeaq msiqlinkav nalindqlim knhlrdimgi pycnyskywy lnhtvtgkts lpkcwlvsng sylnethfsd
dieqqadnmi tellqkeymd rqgktplglv dlfvfstsfy lisiflhlvk ipthrhivgr pcpkphrlnh mgicscglyk
hpgvpvkwkr
(Lassa GP sequence Lineage 4 (NP_694870.1)
SEQ ID NO: 38
mgqivtffqe vphvieevmn ivlialsvla vlkglynfat cglvglvtfl llcgrsctts sdahkknlyd halmsiistf
hlsipnfnqy eamscdfngg kisvqynlsh syagdaanhc gtvangvlqt fmrmawggsy ialdsgrgnw dcimtsyqyl
iiqnttwedh cqfsrpspig ylgllsqrtr diyisrrllg tftwtlsdse gkdtpggycl trwmlieael kcfgntavak
cnekhdeefc dmlrlfdfnk qaiqrlkaea qmsiqlinka vnalindqli mknhlrdimg ipycnyskyw ylnhtttgrt
slpkcwlvsn gsylnethfs ddieqqadnm itemlqkeym erqgktplgl vdlfvfstsf ylisiflhlv kipthrhivg
kscpkphrln hmgicscgly kqpgvpvkwk r
(Lassa GP sequence Lineage 5 (AHC95557.1)
SEQ ID NO: 39
mrnsdfflfd mgqivtffqe vphvieevmn ivlialsila vlkglyniat cgliglvtff llcgrscssn lykgvyelqs
ldlnmetlnm tmplsctknn shhyirvgne tgleltltnt sllnhkfcnl sdahkrnlyd halmsiistf hlsipnfnqy
eamscdfngg kitvqynlsh syagdtakhc gtiangvlqt fmrmawggsy ialdsghgnw dcimtsyqyl iiqnttwedh
cqfsrpspig ylgllsqrtr diyisrrllg tftwtlsdse gnatpggycl trwmlieael kcfgntavak cnekhdeefc
dmlrlfdfnk qaisrlrsea qmsiqlinka vnalindqli mknhlrdimg ipycnyskyw ylnhtvtgrt slpkcwlvsn
gsylnethfs ddieqqadnm itemlqkeym drqgktplgl vdlfvfstsf ylisiflhlv kipthrhivg kpcpkphrln
rmgicscgly kqpgvpvkwk r
(Lassa GP sequence Lineage 6 (AMR44577.1)
SEQ ID NO: 40
mgqiitffqe vphvieevmn ivlialslla ilkgvynvat cgiiglvtfl flcgrscsli ykgsyelqtl elnmetlnmt
mplsctknss hhyirvgnet gleltltnts iinhkfcnlsdahkrnlydh almsilstfh lsipnfnqye amscdfnggk
isvqynlsha yavdaaehcgtvangvlqtf mrmawggsyi aldsgrgnwd cimtsyqyli iqnttwedhc
qfsrpspigylgllsqrtrd iyisrrllgt ftwtlsdseg netpggyclt rwmlieaelk cfgntavakcnekhdeefcd mlrlfdfnkq
aiqrlkseaq msiqlinkav nalindqlim knhlrdmmgipycnyskywy lnhtssgrts lpkcwlvsng sylnethfsd
dieqqadnmi temlqkeyidrqgktplglv dlfvfstsfy lisiflhlik ipthrhivgk pcpkphrlnh
mgicscglykqpgvptrwkr
(Lassa GP sequence Lineage 7 (ANH09740.1)
SEQ ID NO: 41
mgqivtffqe vphvieevmn ivlialslla ilkglynfat cgviglitfl llcgrscsat ykgqyelqtl elnmeslnmt
mplsctknns hhyiragnnt gleltltnts iishkfcnlsdahkknlydh tlmsiittfh lsipnfnqye amscdfnggk
isiqynlshs yagdaaqhcgtvangvlqtf mrmawggsyl aldsgrrgwd ciissyqyli iqnttwddhc
qfsrpspigylgfvsqktrd iyisrrllgt ftwtlsdseg hdmpggyclt rwmlieadlk cfgntavakcnekhdeefcd
mwrlfdfnkq aiqrlkaeaq mniqlinkav nalindqlmm knhlrdimgipycnyskfwy lnntttgrts lprcwlisng
sylnethfsd dieqqadnmi temlqkeymdrqgktplglv dlfvfstsfy litiflhlvk ipthrhivgk pcpkphrlnh
mgicscglykqpgvpvrwkr
Lymphocytic choriomeningitis virus (Clone 13 strain, ABC96001.2)
SEQ ID NO: 42
mgqivtmfea lphiidevin iviivlivit gikavynfat cgifalisfl llagrscgmyglkgpdiykg vyqfksvefd
mshlnltmpn acsannshhy ismgtsglel tfindsiishnfcnltsafn kktfdhtlms ivsslhlsir gnsnykavsc
dfnngitiqy nltfsdaqsaqsqcrtfrgr vldmfrtafg gkymrsgwgw tgsdgkttwc sqtsyqylii
qnrtwenhctyagpfgmsri llsqektkfl trrlagtftw tlsdssgven pggycltkwm ilaaelkcfgntavakcnvn
hdeefcdmlr lidynkaals kfkedvesal hlfkttvnsl isdqllmrnhlrdlmgvpyc nyskfwyleh aktgetsvpk
cwlvtngsyl nethfsdqie qeadnmitemlrkdyikrqg stplalmdll mfstsaylvs iflhlvkipt hrhikggscp
kphrltnkgicscgafkvpg vktvwkrr
Lujo virus (AFP21514.1)
SEQ ID NO: 43
mgqivavfqa ipeilneain iviiviimft likgvfnlyk sglfqlvifl llcgkrcdssllsgfnletv hfnmsllssi pmvseqqhci
qhnhssitfs lltnksdlek cnftrlqavdrvifdlfref hhrvgdfpvt sdlkcshnts yrvieyevtk eslprlqeav
stlfpdlhlsedrflqiqah ddknctglhp lnylrllken sethykvrkl mklfqwslsd etgsplpgghclerwlifas
dikcfdnaai akcnkehdee fedmlrlfdy nkasiaklrg easssinllsgrinaiisdt llmrsslkrl mgipycnytk
fwylnhtklg ihslprcwlv sngsylnetkfthdmedead klltemlkke yvrrqektpi tlmdilmfsv sfymfsvtlc
icnipthrhitglpcpkphr lrkngtcacg ffksinrstg wakh
Machupo virus (AMZ00396.1)
SEQ ID NO: 44
mgqlisffqe ipvflqealn ialvavslia vikgiinlyk sglfqfiffl llagrscsdgtfkiglhtef qsvtltmqrl lanhsnelps
lcmlnnsfyy mkggvntfli rvsdisvltkehdvsiyepe dlgnclnksd sswaihwfsn alghdwlmdp pmlcrnktkr
egsniqfniskaddvrvygk kirngmrhlf rgfhdpceeg rkcyltinqc gdpssldycg tdhlskcqfdhvntlhflvr
skthlnfers lkaffswslt dssgkdmpgg ycleewmlia akmkcfgntavakcnqnhds efcdmlrlfd ynknaiktln
deskkeinll sqtvnalisd nllmknkikelmsipycnyt kfwyvnhtlt gqhtlprcwl irngsylnts efrndwiles
dhlisemlskeyaerqgktp itlvdicfws tvfftaslfl hlvgipthrh lkgeacplph kldsfggcrcgkyprlrkpt iwhrrh
Junin virus (AAB65463.1)
SEQ ID NO: 45
mgqfisfmqe iptflqealn ialvavslia iikgvvnlyk sglfqffvfl alagrscteeafkiglhtef qtvsfsmvgl fsnnphdlpl
lctlnkshly ikggnasfki sfddiavllpeydviiqhpa dmswcsksdd qiwlsqwfmn avghdwyldp pflcrnrtkt
egfifqvntsktginenyak kfktgmhhly reypdscldg klclmkaqpt swpvqcpldh vntlhfltrgkniqlprrsl
kaffswsltd ssgkdtpggy cleewmlvaa kmkcfgntav akcnlnhdsefcdmlrlfdy nknaiktlnd etkkqvnlmg
qtinalisdn llmknkirel msvpycnytkfwyvnhtlsg qhslprcwli knnsylnisd frndwilesd flisemlske
ysdrqgktpltlvdicfwst vfftaslflh lvgipthrhi rgeacplphr lnslggcrcg kypnlkkptvwrrgh

What is claimed is:

1. A recombinant arenavirus glycoprotein comprising an arenavirus glycoprotein ectodomain and a trimerization domain.

2. The recombinant arenavirus glycoprotein of claim 1, wherein said ectodomain comprises an arenavirus GP1/GP2 protein, wherein said arenavirus GP1/GP2 protein comprises a GP1 domain and a GP2 domain.

3. The recombinant arenavirus glycoprotein of claim 2, wherein the C-terminus of the GP1 domain is bound to the N-terminus of the GP2 domain.

4. The recombinant arenavirus glycoprotein of claim 3, wherein said ectodomain further comprises a protease cleavage site, wherein said protease cleavage site is located between said GP1 domain and said GP2 domain.

5. The recombinant arenavirus glycoprotein of claim 2, wherein said arenavirus GP1/GP2 protein is a stabilized arenavirus GP1/GP2 protein.

6. The recombinant arenavirus glycoprotein of claim 5, wherein said stabilized arenavirus GP1/GP2 protein comprises a disulfide bond between a first cysteine amino acid side chain in the GP1 domain and a second cysteine amino acid side chain in the GP2 domain.

7. The recombinant arenavirus glycoprotein of claim 6, wherein said first cysteine amino acid side chain in the GP1 domain is at a position corresponding to position 207 of SEQ ID NO:18 and said second cysteine amino acid side chain in the GP2 domain is at a position corresponding to position 360 of SEQ ID NO:18.

8. The recombinant arenavirus glycoprotein of claim 2, wherein said trimerization domain is bound to the C-terminus of said GP2 domain.

9. The recombinant arenavirus glycoprotein of claim 8, wherein said trimerization domain is covalently bound to said C-terminus of said GP2 domain though a peptide linker.

10. The recombinant arenavirus glycoprotein of claim 9, wherein said peptide linker is from about 5 to about 80 amino acid residues in length.

11. The recombinant arenavirus glycoprotein of claim 9, wherein said peptide linker is from about 8 to about 30 amino acid residues in length.

12. The recombinant arenavirus glycoprotein of claim 1, wherein said trimerization domain is a protein having a molecular weight of about 2 kDa to about 50 kDa.

13. The recombinant arenavirus glycoprotein of claim 12, wherein said trimerization domain is a protein having a molecular weight of about 3 kDa to about 30 kDa.

14. The recombinant arenavirus glycoprotein of claim 1, wherein said trimerization domain is a leucine zipper, 1NOG, Fibritin, or GCN4.

15. The recombinant arenavirus glycoprotein of claim 14, wherein said trimerization domain is 1NOG.

16. The recombinant arenavirus glycoprotein of claim 1, wherein said ectodomain comprises:
an arginine at a position corresponding to position 258 of SEQ ID NO:18,
an arginine at a position corresponding to position 259 of SEQ ID NO:18, and
a proline at a position corresponding to position 329 of SEQ ID NO:18.

17. The recombinant arenavirus glycoprotein of claim 1, wherein said ectodomain comprises:
a phenylalanine at a position corresponding to position 193 of SEQ ID NO:18,
a leucine at a position corresponding to position 211 of SEQ ID NO:18,
a leucine at a position corresponding to position 339 of SEQ ID NO:18, and
a leucine at a position corresponding to position 354 of SEQ ID NO:18.

18. The recombinant arenavirus glycoprotein of claim 1, wherein said ectodomain further comprises:
a proline at a position corresponding to position 128 of SEQ ID NO:18, or
an isoleucine, leucine or valine at a position corresponding to position 305 of SEQ ID NO:18.

19. The recombinant arenavirus glycoprotein of claim 1, wherein said ectodomain comprises an amino acid sequence having a sequence identify of at least 80% to the amino acid sequence of SEQ ID NO:27.

20. The recombinant arenavirus glycoprotein of claim 2, wherein said ectodomain further comprises a signal peptide.

21. The recombinant arenavirus glycoprotein of claim 20, wherein said signal peptide is covalently bound to the N-terminus of the GP1 domain.

22. The recombinant arenavirus glycoprotein of claim 20, wherein said signal peptide comprises the sequence of SEQ ID NO:2.

23. The recombinant arenavirus glycoprotein of claim 1, wherein said ectodomain is non-glycosylated.

24. The recombinant arenavirus glycoprotein of claim 1, wherein said ectodomain is glycosylated.

25. The recombinant arenavirus glycoprotein of claim 24, wherein the ectodomain is glycosylated at one or more amino acid residues at positions corresponding to 79, 89, 99, 119, 365 and 373 of SEQ ID NO:18.

26. The recombinant arenavirus glycoprotein of claim 1, wherein said ectodomain is not glycosylated at one or more amino acid residues at positions corresponding to 390 or 395 of SEQ ID NO:18.

27. A glycoprotein trimer comprising three of the recombinant arenavirus glycoproteins of claim 1, wherein said three recombinant arenavirus glycoproteins are bound by non-covalent attachment of the trimerization domains.

28. A nucleic acid encoding the recombinant arenavirus glycoprotein of claim 1.

29. The nucleic acid of claim 28, further comprising a vector.

30. A cell comprising the recombinant arenavirus glycoprotein of claim 1 or 2 the glycoprotein trimer of claim 27.

31. A cell comprising the nucleic acid of claim 28.

32. The cell of claim 30, wherein the cell is a human cell.

33. A vaccine composition comprising the recombinant arenavirus glycoprotein of claim 1 and a pharmaceutically acceptable excipient.

34. A vaccine composition comprising the glycoprotein trimer of claims 27 and a pharmaceutically acceptable excipient.

35. The vaccine composition of claim 33, further comprising an adjuvant.

36. A method of treating or preventing a viral disease in a subject in need of such treatment or prevention, said method comprising administering a therapeutically or prophylactically effective amount of the recombinant arenavirus glycoprotein of claim 1 to said subject.

37. A method of treating or preventing a viral disease in a subject in need of such treatment or prevention, said method comprising administering a therapeutically or prophylactically effective amount of the glycoprotein trimer of claim 27 to said subject.

38. The method of claim 36, wherein said viral disease is caused by Lymphocytic choriomeningitis virus (LCMV), Junin virus, Machupo virus, Lassa virus, Guanarito virus, Sabia virus, Chapare virus, Whitewater Arroyo virus, or Lujo virus.

39. A method for immunizing a subject susceptible to a viral disease, comprising administering the recombinant arenavirus glycoprotein of claim 1 to a subject under conditions such that antibodies directed to said arenavirus glycoprotein or a fragment thereof are produced.

40. A method for immunizing a subject susceptible to a viral disease, comprising administering the glycoprotein trimer of claim 27 to a subject under conditions such that antibodies directed to said glycoprotein trimer or a fragment thereof are produced.

41. A method of diagnosing arenavirus infection in a subject, the method comprising: (a) contacting a biological sample obtained from the subject with the recombinant arenavirus glycoprotein of claim 1, and (b) detecting binding of one or more antibodies to said recombinant arenavirus glycoprotein, thereby diagnosing arenavirus infection in said subject.

42. A method of diagnosing arenavirus infection in a subject, the method comprising: (a) contacting a biological sample obtained from the subject with the glycoprotein trimer of claim 27, and (b) detecting binding of one or more antibodies to said glycoprotein trimer, thereby diagnosing arenavirus infection in said subject.

43. A method for evaluating effectiveness of an arenavirus vaccine in a subject, the method comprising: (a) contacting a biological sample from a subject who has been administered with the vaccine composition of claim 33 or 34, (b) detecting antibodies in the biological sample that bind to the recombinant arenavirus glycoprotein or glycoprotein trimer, and (c) performing quantitative and qualitative analysis of the antibodies detected in the biological sample, thereby evaluating effectiveness of the arenavirus vaccine in the subject.


SEQUENCE LISTING (45 sequences)

SEQ ID NO: 1 - protein, 624 aa, Artificial Sequence
  Synthetic polypeptide
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVDLEGGSPVVEVQGTIDELNSFIGYALVLSRWDDIRNDLFRIQNDLFVLGEDVSTGGKGRTVTREMIDYLEARVKEMKAEIGKIELFVVPGGSVESASLHMARAVSRRLERRIVAASKLTEINKNVLIYANRLSSILFMHALISNKRLNIPEKIWALEVDDDDKAGWSHPQFEKGGGSGGGSGGGSWSHPQFEK

SEQ ID NO: 2 - protein, 58 aa, Lassa virus
MGQIVTFFQEVPHVIEEVMNIVLIALSVLAVLKGLYNFATCGLVGLVTFLLLCGRSCT

SEQ ID NO: 3 - protein, 374 aa, Artificial Sequence
  Synthetic polypeptide
TSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVD

SEQ ID NO: 4 - protein, 13 aa, Artificial Sequence
  Synthetic polypeptide
MERQGKTPLGLVD

SEQ ID NO: 5 - protein, 149 aa, Thermoplasma acidophilum
PVVEVQGTIDELNSFIGYALVLSRWDDIRNDLFRIQNDLFVLGEDVSTGGKGRTVTREMIDYLEARVKEMKAEIGKIELFVVPGGSVESASLHMARAVSRRLERRIVAASKLTEINKNVLIYANRLSSILFMHALISNKRLNIPEKIWA

SEQ ID NO: 6 - protein, 5 aa, Artificial Sequence
  Synthetic polypeptide
DDDDK

SEQ ID NO: 7 - protein, 8 aa, Artificial Sequence
  Synthetic polypeptide
WSHPQFEK

SEQ ID NO: 8 - protein, 15 aa, Artificial Sequence
  Synthetic polypeptide
GLNDIFEAQKIEWHE

SEQ ID NO: 9 - protein, 8 aa, Artificial Sequence
  Synthetic polypeptide
LEVLFQGP

SEQ ID NO: 10 - protein, 465 aa, Artificial Sequence
  Synthetic polypeptide
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEYLPETGLVDLEVDDDDKAGWSHPQFEKGGGSGGGSGGGSWSHPQFEK

SEQ ID NO: 11 - protein, 184 aa, Artificial Sequence
  Synthetic polypeptide
GGGGGSGSPVVEVQGTIDELNSFIGYALVLSRWDDIRNDLFRIQNDLFVLGEDVSTGGKGRTVTREMIDYLEARVKEMKAEIGKIELFVVPGGSVESASLHMARAVSRRLERRIVAASKLTEINKNVLIYANRLSSILFMHALISNKRLNIPEKIWALEVHHHHHHGSGGLNDIFEAQKIEWHE

SEQ ID NO: 12 - protein, 6 aa, Artificial Sequence
  Synthetic polypeptide
LVDLEV

SEQ ID NO: 13 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 14 - protein, 5 aa, Artificial Sequence
  Synthetic polypeptide
LPETG

SEQ ID NO: 15 - protein, 5 aa, Artificial Sequence
  Synthetic polypeptide
  misc_feature (3)..(3): Xaa can be any naturally occurring amino acid
LPXTG

SEQ ID NO: 16 - protein, 32 aa, Artificial Sequence
  Synthetic polypeptide
MLQKEYMERQGKTPLGLVDLEGGSPVVEVQGT

SEQ ID NO: 17 - protein, 25 aa, Artificial Sequence
  Synthetic polypeptide
MLQKEYLPETGGGGGSGSVVEVQGT

SEQ ID NO: 18 - protein, 419 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEY

SEQ ID NO: 19 - protein, 7 aa, Artificial Sequence
  Synthetic polypeptide
ENLYFQG

SEQ ID NO: 20 - protein, 7 aa, Artificial Sequence
  Synthetic polypeptide
ENLYFQS

SEQ ID NO: 21 - protein, 6 aa, Artificial Sequence
  Synthetic polypeptide
LVPRGS

SEQ ID NO: 22 - protein, 7 aa, Artificial Sequence
  Synthetic polypeptide
  MISC_FEATURE (1)..(1): Hydrophobic amino acid
  MISC_FEATURE (3)..(3): Hydrophobic amino acid
  MISC_FEATURE (7)..(7): Hydrophobic amino acid
XRXKRRX

SEQ ID NO: 23 - protein, 6 aa, Artificial Sequence
  Synthetic polypeptide
RRSKLL

SEQ ID NO: 24 - protein, 4 aa, Artificial Sequence
  Synthetic polypeptide
RRLA

SEQ ID NO: 25 - protein, 4 aa, Artificial Sequence
  Synthetic polypeptide
RKLM

SEQ ID NO: 26 - protein, 4 aa, Artificial Sequence
  Synthetic polypeptide
RSLK

SEQ ID NO: 27 - protein, 361 aa, Artificial Sequence
  Synthetic polypeptide
TSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEY

SEQ ID NO: 28 - protein, 199 aa, Artificial Sequence
  Synthetic polypeptide
TSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRR

SEQ ID NO: 29 - protein, 162 aa, Artificial Sequence
  Synthetic polypeptide
RRGTFTWTLSDSEGKDTPGGYCLTRWMLIEAELKCFGNTAVAKCNEKHDEEFCDMLRLFDFNKQAIQRLKAPAQTSIQLINKAVNALINDQLIMKNHLRDIMCIPYCNYSKYWYLNHTTTGRTSLPKCWLVSNGSYLNETHFSDDIEQQADNMITEMLQKEY

SEQ ID NO: 30 - protein, 4 aa, Artificial Sequence
  Synthetic polypeptide
RRRR

SEQ ID NO: 31 - protein, 4 aa, Artificial Sequence
  Synthetic polypeptide
RRLL

SEQ ID NO: 32 - protein, 22 aa, Artificial Sequence
  Synthetic polypeptide
MLQKEYMERQGGGSPVVEVQGT

SEQ ID NO: 33 - protein, 25 aa, Artificial Sequence
  Synthetic polypeptide
MLQKEYMERQGKTPAPAPVVEVQGT

SEQ ID NO: 34 - protein, 29 aa, Artificial Sequence
  Synthetic polypeptide
MLQKEYMERQGKTPAPAPGGSPVVEVQGT

SEQ ID NO: 35 - protein, 430 aa, Lassa virus
MGQIITFFQEVPHVIEEVMNIVLIALSLLAILKGLYNIATCGIIGLVAFLFLCGKSCSLTLKGGYELQTLELNMETLNMTMPLSCTKNSSHHYIRVGNETGLELTLTNTSIINHKFCNLSDAHKKNLYDHALMSIISTFHLSIPNFNQYEAMSCDFNGGKISVQYNLSHSYAGDAAEHCGTVANGVLQTFMRMAWGGRYIALDSGKGNWDCIMTSYQYLIIQNTTWEDHCQFSRPSPIGYLGLLSQRTRDIYISRRLLGTFTWTLSDSEGNETPGGYCLTRWMLIEAELKCFGNTAVAKCNEKHDEEFCDMLRLFDFNKQAIRRLKAEAQMSIQLINKAVNALINDQLIMKNHLRDIMGIRQGKTPLGLVDIFIFSTSFYLISIFLHLIKIPTHRHIVGKPCPKPHRLNHMGVCSCGLYKHPGVPTKWKR

SEQ ID NO: 36 - protein, 490 aa, Lassa virus
MGQIITFFQEVPHVIEEVMNIVLIALSLLAILKGIYNVATCGLFGLVSFLLLCGRSCSTTYKGVYELQTLELDMASLNMTMPLSCTKNNSHHYIMVGNETGLELTLTNTSIINHKFCNLSDAHKKNLYDHALMSIISTFHLSIPNFNQYEAMSCDFNGGKISVQYNLSHTYAVDAANHCGTIANGVLQTFMRMAWGGSYIALDSGKGSWDCIMTSYQYLIIQNTTWEDHCQFSRPSPIGYLGLLSQRTRDIYISRRLLGTFTWTLSDSEGNETPGGYCLTRWMLIEAELKCFGNTAVAKCNEKHDEEFCDMLRLFDFNKQAIRRLKTEAQMSIQLINKAVNALINDQLIMKNHLRDIMGIPYCNYSKYWYLNHTVTGRTSLPRCWLVSNGSYLNETHFSDDIEQQADNMITELLQKEYIDRQGKTPLGLVDLFVFSTSFYLISIFLHLIKIPTHRHVIGKPCPKPHRLNHMGICSCGLYKHPGVPVKWKR

SEQ ID NO: 37 - protein, 490 aa, Lassa virus
MGQIITFFQEVPHVIEEVMNIVLIALSLLAILKGIYNIATCGLFGLISFLLLCGRSCSTTYKGVYELQTLELDMANLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAVDAAGHCGTIANGVLQTFMRMAWGGSYIALDSGKGNWDCIMTSYQYLVIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIRRLKAEAQMSIQLINKAVNALINDQLIMKNHLRDIMGIPYCNYSKYWYLNHTVTGKTSLPKCWLVSNGSYLNETHFSDDIEQQADNMITELLQKEYMDRQGKTPLGLVDLFVFSTSFYLISIFLHLVKIPTHRHIVGRPCPKPHRLNHMGICSCGLYKHPGVPVKWKR

SEQ ID NO: 38 - protein, 431 aa, Lassa virus
MGQIVTFFQEVPHVIEEVMNIVLIALSVLAVLKGLYNFATCGLVGLVTFLLLCGRSCTTSSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 39 - protein, 501 aa, Lassa virus
MRNSDFFLFDMGQIVTFFQEVPHVIEEVMNIVLIALSILAVLKGLYNIATCGLIGLVTFFLLCGRSCSSNLYKGVYELQSLDLNMETLNMTMPLSCTKNNSHHYIRVGNETGLELTLTNTSLLNHKFCNLSDAHKRNLYDHALMSIISTFHLSIPNFNQYEAMSCDFNGGKITVQYNLSHSYAGDTAKHCGTIANGVLQTFMRMAWGGSYIALDSGHGNWDCIMTSYQYLIIQNTTWEDHCQFSRPSPIGYLGLLSQRTRDIYISRRLLGTFTWTLSDSEGNATPGGYCLTRWMLIEAELKCFGNTAVAKCNEKHDEEFCDMLRLFDFNKQAISRLRSEAQMSIQLINKAVNALINDQLIMKNHLRDIMGIPYCNYSKYWYLNHTVTGRTSLPKCWLVSNGSYLNETHFSDDIEQQADNMITEMLQKEYMDRQGKTPLGLVDLFVFSTSFYLISIFLHLVKIPTHRHIVGKPCPKPHRLNRMGICSCGLYKQPGVPVKWKR

SEQ ID NO: 40 - protein, 490 aa, Lassa virus
MGQIITFFQEVPHVIEEVMNIVLIALSLLAILKGVYNVATCGIIGLVTFLFLCGRSCSLIYKGSYELQTLELNMETLNMTMPLSCTKNSSHHYIRVGNETGLELTLTNTSIINHKFCNLSDAHKRNLYDHALMSILSTFHLSIPNFNQYEAMSCDFNGGKISVQYNLSHAYAVDAAEHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGNETPGGYCLTRWMLIEAELKCFGNTAVAKCNEKHDEEFCDMLRLFDFNKQAIQRLKSEAQMSIQLINKAVNALINDQLIMKNHLRDMMGIPYCNYSKYWYLNHTSSGRTSLPKCWLVSNGSYLNETHFSDDIEQQADNMITEMLQKEYIDRQGKTPLGLVDLFVFSTSFYLISIFLHLIKIPTHRHIVGKPCPKPHRLNHMGICSCGLYKQPGVPTRWKR

SEQ ID NO: 41 - protein, 490 aa, Lassa virus
MGQIVTFFQEVPHVIEEVMNIVLIALSLLAILKGLYNFATCGVIGLITFLLLCGRSCSATYKGQYELQTLELNMESLNMTMPLSCTKNNSHHYIRAGNNTGLELTLTNTSIISHKFCNLSDAHKKNLYDHTLMSIITTFHLSIPNFNQYEAMSCDFNGGKISIQYNLSHSYAGDAAQHCGTVANGVLQTFMRMAWGGSYLALDSGRRGWDCIISSYQYLIIQNTTWDDHCQFSRPSPIGYLGFVSQKTRDIYISRRLLGTFTWTLSDSEGHDMPGGYCLTRWMLIEADLKCFGNTAVAKCNEKHDEEFCDMWRLFDFNKQAIQRLKAEAQMNIQLINKAVNALINDQLMMKNHLRDIMGIPYCNYSKFWYLNNTTTGRTSLPRCWLISNGSYLNETHFSDDIEQQADNMITEMLQKEYMDRQGKTPLGLVDLFVFSTSFYLITIFLHLVKIPTHRHIVGKPCPKPHRLNHMGICSCGLYKQPGVPVRWKR

SEQ ID NO: 42 - protein, 498 aa, Lymphocytic choriomeningitis virus
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 43 - protein, 454 aa, Lujo virus
MGQIVAVFQAIPEILNEAINIVIIVIIMFTLIKGVFNLYKSGLFQLVIFLLLCGKRCDSSLLSGFNLETVHFNMSLLSSIPMVSEQQHCIQHNHSSITFSLLTNKSDLEKCNFTRLQAVDRVIFDLFREFHHRVGDFPVTSDLKCSHNTSYRVIEYEVTKESLPRLQEAVSTLFPDLHLSEDRFLQIQAHDDKNCTGLHPLNYLRLLKENSETHYKVRKLMKLFQWSLSDETGSPLPGGHCLERWLIFASDIKCFDNAAIAKCNKEHDEEFCDMLRLFDYNKASIAKLRGEASSSINLLSGRINAIISDTLLMRSSLKRLMGIPYCNYTKFWYLNHTKLGIHSLPRCWLVSNGSYLNETKFTHDMEDEADKLLTEMLKKEYVRRQEKTPITLMDILMFSVSFYMFSVTLCICNIPTHRHITGLPCPKPHRLRKNGTCACGFFKSINRSTGWAKH

SEQ ID NO: 44 - protein, 496 aa, Machupo virus
MGQLISFFQEIPVFLQEALNIALVAVSLIAVIKGIINLYKSGLFQFIFFLLLAGRSCSDGTFKIGLHTEFQSVTLTMQRLLANHSNELPSLCMLNNSFYYMKGGVNTFLIRVSDISVLTKEHDVSIYEPEDLGNCLNKSDSSWAIHWFSNALGHDWLMDPPMLCRNKTKREGSNIQFNISKADDVRVYGKKIRNGMRHLFRGFHDPCEEGRKCYLTINQCGDPSSLDYCGTDHLSKCQFDHVNTLHFLVRSKTHLNFERSLKAFFSWSLTDSSGKDMPGGYCLEEWMLIAAKMKCFGNTAVAKCNQNHDSEFCDMLRLFDYNKNAIKTLNDESKKEINLLSQTVNALISDNLLMKNKIKELMSIPYCNYTKFWYVNHTLTGQHTLPRCWLIRNGSYLNTSEFRNDWILESDHLISEMLSKEYAERQGKTPITLVDICFWSTVFFTASLFLHLVGIPTHRHLKGEACPLPHKLDSFGGCRCGKYPRLRKPTIWHRRH

SEQ ID NO: 45 - protein, 485 aa, Junin virus
MGQFISFMQEIPTFLQEALNIALVAVSLIAIIKGVVNLYKSGLFQFFVFLALAGRSCTEEAFKIGLHTEFQTVSFSMVGLFSNNPHDLPLLCTLNKSHLYIKGGNASFKISFDDIAVLLPEYDVIIQHPADMSWCSKSDDQIWLSQWFMNAVGHDWYLDPPFLCRNRTKTEGFIFQVNTSKTGINENYAKKFKTGMHHLYREYPDSCLDGKLCLMKAQPTSWPVQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICFWSTVFFTASLFLHLVGIPTHRHIRGEACPLPHRLNSLGGCRCGKYPNLKKPTVWRRGH